(19) United States
(12) Patent Application Publication
(10) Pub. No.: US 2026/0278151 A1
(43) Pub. Date: Sep. 17, 2026

(54) MEANS FOR INTERACTION OF A MEDICAL DEVICE CONNECTIVITY SYSTEM WITH NON-MEDICAL SOFTWARE TO ENABLE AUTOMATIC ACCESS OF REMOTE MONITORING DATA AND PROGRAMMING DATA BY RELEVANT STAKEHOLDERS

(71) Applicant: BIOTRONIK SE & Co. KG, Berlin (DE)

(72) Inventors: Adrian Kimber, Lake Forest, IL (US); David McIntosh, Wilsonville, OR (US); James Horton, Portland, OR (US); Karsten Kreutziger, Berlin (DE); Kevin Gehle, Minneapolis, MN (US); Lauren Kraiter, Tigard, OR (US); Andrew B. Kibler, Lake Oswego, OR (US); Sean Slee, Tigard, OR (US); Min Qu, Portland, OR (US); Thorsten Wenzlaff, Berlin (DE)

(21) Appl. No.: 19/168,593

(22) PCT Filed: Mar. 26, 2024
(86) PCT No.: PCT/EP2024/058034

§ 371 Date: Sep. 24, 2025

Related U.S. Application Data
(60) Provisional application No. 63/456,156, filed on Mar. 31, 2023.

(30) Foreign Application Priority Data
May 4, 2024 (EP) 23171568.1

(51) Int. Cl. G06F 21/62; G06F 21/60; G16H 10/60

(52) U.S. Cl. CPC G06F 21/6245; G06F 21/602; G16H 10/60

(57) ABSTRACT

A system for medical data processing is provided, the system comprising means for receiving encrypted medical data from a secure medical device server; means for decrypting the medical data at least partly; and means for providing the at least partly decrypted data to a database.

The invention relates to systems, methods, computer programs, and user interfaces for processing of medical data such as to provide an interface between a secure medical device database and an (external user) database. For example, (auto-generated) reports may be provided for export into the external user database(s). A distributed medical device connectivity system (herein also simply called “system”) may thus be enabled to automatically and seamlessly export data to a non-medical device system with a number of user interfaces.

Many medical devices (both external and implantable devices) have the ability to capture and store medical data (patient and device data). Such medical data typically relates to patient status, medical event detection and therapy, etc. This data can be viewed during in-person sessions, e.g., at the treating doctor's office, via use of a specialized viewing/programming device. Some systems comprising said specialized devices have the ability to transmit this data remotely for use in viewing in a dedicated user interface provided by the device manufacturer. A few such vendors have the ability to export this data for viewing in an external user interface, though this typically is expensive both in terms of the cost and time for building such a connection, as well as for maintaining the connection going forward. The current paradigm is that each such connectivity solution must be customized for the needs of that particular installation, requiring a custom IT project. As a custom IT project, each such system requires unique attention to maintain its functionality going forward. Further, additional software licenses frequently must be acquired to operate the system. Known systems do not have the ability to export data to an external interface without a standalone project (and its associated costs) to build the connection. Therefore, no known vendor's system offers consolidation of all users' workflows into one turnkey, integrated and automatic system.

Therefore, there is still a need to further improve distributed medical device connectivity systems.

The various aspects described herein may meet the above need at least in part.

A first aspect of the invention is described in the following:

According to one aspect of the invention, a system for medical data processing is provided. The system comprises means for receiving encrypted medical data from a secure medical device database, means for decrypting the medical data at least partly, and means for providing the at least partly-decrypted data to a database.

This may ensure that user(s) have uninterrupted access to basic patient profile data needed to provide a proactive care service (e.g., enough data to identify a patient, identify their medical device, e.g., an implant, or assign a patient to the medical device). Specifically, the system may operate using multiple different data reporters (medical devices and non-medical devices, e.g., devices running a patient app as described herein, wherein various devices may relate to the same or different patients). Further, various different data consumers/recipients may be served. The system may thus provide a single, turnkey source for an end-to-end integrated data network/system without the requirement for a stand-alone, custom IT project to integrate the system into a recipient's existing IT-system. Instead, the medical data from the various sources received from the secure medical device database may be transformed and provided to a second database such that the database may be easily accessed by various data consumers, e.g., using a web-based log-in procedure.

In one example, the means for decrypting may be adapted such that the decrypted medical data comprises a file structure with key-value pairs, preferably in a human-readable language. Additionally or alternatively, the means for decrypting may be adapted such that the decrypted data are free of privacy-sensitive information. This may advantageously improve legibility, processibility, and/or accessibility of the data and thereby reduce the costs and workload associated with processing data comprising privacy-sensitive information from a regulatory standpoint. The means for decrypting may, for example, be adapted to provide the decrypted data in a JSON-file format and/or any other similar file format.

In an example, the means for receiving is further configured to receive (e.g., objective and/or subjective) data from at least one external device, e.g., associated with one or more patients. Objective data may, e.g., comprise any parameter measured by the medical device and/or the external device, an indication whether said device is switched off or on, the operation mode of said device, a timestamp, etc.—data that do not rely on any subjective judgement by a user (e.g., the patient or the attending health professional). Subjective data, in contrast, may, e.g., comprise patient- and/or health professional-reported data, e.g., patient's pain scores, measures for comfort, and/or any further data collected, e.g., by questionnaires issued to the patient and/or the health professional-data that rely on subjective judgement by the user (e.g., the patient or the attending health professional). The collection of objective as well as subjective data may occur either on demand, e.g., measuring an objective parameter directly, and/or issuing a questionnaire to the patient requesting a subjective parameter, e.g., their current pain score. The on-demand request may, e.g., be triggered when at least one parameter of the medical data lies without a predetermined “safe” range and/or indicates the need to respond to an underlying issue in any other way. Hence, the second database may be supplemented with such additional data as well, such that more comprehensive data exists on a patient.

The objective and/or subjective data may for example be collected (automatically) by wearable devices, e.g., smart watches etc., e.g., via triggered collection of subjective and health data from non-medical application(s).

The objective and/or subjective data may, for example, comprise patient-reported data, health professional-reported data, and/or device-reported data. Hence, various data sources may be integrated into the system.

The system may, for example, further comprise means for triggering an alert, based at least in part on the medical data, and/or the subjective and/or objective data. This may improve remote proactive care of the patient by monitoring critical parameters, e.g., ensuring that they stay within predetermined, device- and/or patient-specific limits. These limits may define trigger-values, at which an alert may be triggered, when a monitored parameter is below a lower limit or above an upper limit. Such alert may for example comprise a simple notification to any component of the system and/or any user thereof and/or comprise instructions to the system and or any component thereof on how to resolve the underlying issue (automatically).

The means for triggering the alert may be implemented within that (part of the) system that also decrypts and provides the medical data to a second database. Hence, alerts may be triggered directly at the point where the data from the various data sources meets. Additionally or alternatively, the means for triggering the alert may also be implemented in the second database and/or a data consumer accessing the data in the second database.

The system may, e.g., further comprise means for receiving a first data request from the second database, e.g., via the user-interface. This may be an advantageous first step of targeted data interrogation, e.g., of the medical device, the secure medical device database or any other component described herein. Such first data request may for example be issued by a user of the system via the second database, e.g., via a portal backend as described herein.

The system may, for example, further comprise means for sending a second data request to the secure medical device database, preferably based at least in part on the first data request. For example (that part of) the system that decrypts and provides the medical data to the second database may receive the first data request and, in response thereto, send a corresponding second data request to the secure medical device database (that, in turn, may be in communication with a suitable source, e.g. an implant or other medical device, for that data). Such requests may thus be suitable to request data from wherever they are stored within the system as described herein, facilitating the use of the system.

The system may, for example, further comprise at least one means for pushing, in particular automatically pushing, medical data from the secure medical device database to the second database. For example, (a part of) the system may push and/or automatically provide medical data (and/or further data, e.g., personal patient data) from the secure medical database to the second database, either on request or without request.

The system may, e.g., further comprise means for providing medical data at least partly based on the second data request. For example, in response to sending the second data request to the secure medical device database, the medical device database may deliver that data that may then, upon decryption, in turn be provided to the database.

Thereby, the system may provide the medical data on-demand, in their original form or, e.g., in form of pre-processed report (sheets). This may improve user friendliness and efficiency of the system. Each user may access the full range of medical data available with a simple interface, e.g., web-based, that allows access to the system which in turn provides an interface to the secure medical device database.

The encrypted medical data may, for example, comprise measurement and/or operational data of an at least partly implantable medical device. Thereby, the medical data may allow to fully assess the relevant parameters concerning patient state and/or device state, e.g., representing the delivered therapy. Thus, an all-in-one medical data management system may be provided, thereby significantly improving the efficiency of remote care, remote monitoring, and/or remote programming of medical devices.

The system may, for example, further comprise the secure medical device database, wherein the secure medical device database is configured to receive medical data from one or more medical devices. This allows for well-aligned operation of the secure medical device database and the rest of the system as described herein, including seamless data transmission, processing, and/or export as required. This may improve the automatization of the system and reduce the required amount of work done by involved users.

The secure medical device database may, for example, comprise means for data processing configured to automatically provide a follow-up report to the second database upon a program change of the one or more medical devices. A medical device may be programmed including a program change via remote programming through the system described herein (e.g., via an interface to the second database in communication with the system) and/or by a clinician programmer which relies on being brought close to the medical device for establishing a programming connection, for example, via Bluetooth. In either case, the system may issue a program session report as described herein to provide all authorized users of the system with relevant information on the newly programmed medical device, e.g., the new settings of the device in comparison to the previous settings. This may be done directly by (that part of) the system that decrypts the data, particularly in case the program change is implemented via the system. In some examples, the report may be generated and/or triggered e.g., by the medical device and/or the secure medical device database, which then causes the system to issue a corresponding report.

The system may, e.g., further comprise means for data processing configured for processing the medical data to diagnostically identify issues and/or provide visualization data for visualization of the medical data. This may occur in form of configurable (e.g., by the user interface to the second database) and/or standardized reports. The processing may be performed at least partly before and/or after decryption.

The means for data processing may increase the degree of automatization of this system, facilitating the use of the system and improving the accuracy, reliability, and efficiency of the system while reducing the workload of the involved users, e.g., clinicians, at the same time. Automatically identifying issues, for example, related to the patient's health and/or by visualizing the data for the user to identify trends, issues, etc. more easily may be particularly beneficial in this context. The means for data processing may particularly facilitate access by a simple user interface that may e.g., be web-based.

The system may, e.g., further comprise the second database. Additionally or alternatively, it may comprise a user-interface for outputting at least a part of the data in the second database, preferably comprising decrypted and/or processed medical data, patient management data, and/or device-specific data. Additionally or alternatively, the system may comprise at least one medical device, and/or at least one relay device, wherein the relay device operably connects the at least one medical device and a secure medical device server, e.g., comprising the secure medical device database.

The relay device may for example be a patient remote or patient's mobile phone running a respective application enabling the mobile phone to function as a relay device connecting the patient's medical device(s) with the system, e.g., the secure medical device server.

Integrating all these components into one common system may further improve inter-component data exchange, effectively improving the system efficiency and reliability.

Another example according to the first aspect relates to a method for medical data processing, the method comprising the steps of receiving encrypted medical data from a secure medical device database, decrypting the medical data at least partly, and providing the at least partly decrypted data to a second database.

A further example relates to a computer program comprising instructions to execute the steps of the method described herein.

Said method and computer program yield the benefits mentioned herein in reference to the system, which may e.g., execute the steps of the method.

A second aspect of the invention is described in the following:

According to the second aspect, a method for automated patient treatment sequencing is provided. The method comprises the following steps: a) receiving medical data from at least one first medical device, b) defining a patient stage within a predetermined patient treatment sequence, based at least in part on the medical data, and repeating the steps a) and b) for further medical data from the at least one first medical device and/or at least one second medical device.

Hence, the patient may, in some examples, be continuously tracked through various stages based on medical data received from various medical devices. Such automated patient treatment sequencing may allow for automated pain patient journey tracking, for example, wherein the patient journey may be seen as their progress through the treatment sequence. The steps of the predetermined patient treatment sequence may be known from the beginning of the sequence, for example based on knowing the patient, the location of the patient, the attending health professional, and/or the potential medical device the patient might receive when identified as a suitable for receiving such device, etc. The treatment sequence may comprise predetermined stages and/or phases comprising several stages. The phases may, e.g., relate to a candidate phase comprising the registration stage and the registration validation stage, a pre-trial/introduction phase comprising multiple stages (e.g., a psychologist/physician consultation for pre-trial clearance, a pre-trial authorization stage, etc.), a trial phase (comprising, e.g., a medical intervention and trial period stage and a trial follow-up and outcome stage, a pre-implant authorization stage, an implantation stage, and a post-operation follow-up stage), and a long-term follow-up phase comprising, e.g., a continuous automatic data monitoring stage, a trigger monitoring stage, a support/follow-up reporting stage, etc. Such sequencing and/or tracking may advantageously assist in patient identification, access, care reimbursement, and providing improved long-term care for patients.

One of the early aspects is the identification of patient candidates. By using automated tracking, healthcare providers can quickly and accurately identify patients who may be candidates for therapy, such as, e.g., those with chronic pain conditions within the indications of a specific medical treatment considering SCS in this example, and who have exhausted earlier stages of medical care. Hence, for these patients for whom medical data has been received a change in their stage may be determined, e.g., from a generic or default stage to a therapy candidate stage. Depending, for example, on further medical data received, the patient may then be identified as suitable for therapy and the stage may be updated to a stage of the therapy trial phase (e.g., medical intervention and trial period stage). This helps to ensure that patients receive the appropriate treatment as soon as possible, which can improve their quality of life and outcomes. Moreover, it reduces the formalistic burden on the medical staff that do not need to pro-actively switch the monitoring stages themselves which may be burdensome, and which may sometimes even be overlooked. Further, it may increase the consistency of the assessment of the patient's health state independently from the attending health professional.

Once a patient is identified as a candidate for a selected therapy, tracking can also be used to monitor their progress throughout the therapy trial phase. This may include tracking symptoms, side effects, and other important data that can help healthcare providers determine the effectiveness of the therapy. By having this information easily accessible and organized, clinicians/healthcare providers can make more informed decisions about the therapy and potential long-term use by the patient, thereby ensuring the care given is the right fit for a patient's needs.

Patient tracking can also be used to monitor the post-implantation phase of the therapy. This includes tracking the patient's recovery, any complications that may arise, and the long-term effectiveness of the therapy. This can help healthcare providers identify any issues early on and make adjustments as needed to optimize the therapy for the patient. Additionally, automated tracking can also help with follow-up care and monitoring, making sure the patient receives the best care possible.

In summary, automated patient journey tracking helps identifying patient candidates, monitor therapy trials, and track post-implantation recovery and long-term behavior, which ultimately improve the patient's quality of life and outcomes, and help providers make the best decisions for the patient. Further, it enables a dedicated type of monitoring that takes into account the specific predetermined phase the patient is currently in, such that patient monitoring can be improved.

The systems and methods described herein may generally optimize the above steps to ultimately improve a physician's ability to care for their patients and improve long term outcomes for patients selected for a specific treatment sequence/path (particularly pain patients receiving a SCS).

The medical devices described herein may, e.g., comprise pacemakers, other cardiovascular implants, and/or spinal cord stimulators (SCS), but also any further implants, etc.

The first and second medical device may, e.g., comprise an implantable medical device (like SCSs etc.) and/or comprise, e.g., a trial medical device and/or a permanent implant of the patient.

The automatic staging may pose a helpful reference point for patient assessment. The current stage of the patient may serve as an objective basis for the attending health professional, e.g., to assess which therapeutic steps to perform next, to adjust the programming of implantable medical devices, to understand the medical data provided during the remote monitoring session, etc. Further, the system that is itself executing the method described herein may execute further steps automatically, and based at least partly on the current stage of the patient, e.g., in a predetermined way.

In an example, the at least one first and second medical devices comprises an implant, e.g., one of the examples named herein, and the method further comprises receiving medical data from at least one further device before implantation of the at least one first and second medical devices. Preferably, defining the patient stage before implantation is based on the medical data from the at least one further device.

This may help to facilitate clinical ease of use and communication on process status and improve and synchronize patient care, treatment access, and outcome quality across diverse caregiver and assistance groups. Further, this allows a broader range of data to enter the system, allowing for more reliable number/fact-based decisions in patient care.

The medical data from at least one further device may, e.g., be objective data automatically recorded by the further device or subjective data, e.g., patient-reported and/or clinician-reported data, e.g., requested via surveys, e.g., provided to the user via a portal interface and/or an app on the patient's mobile phone and/or another suitable device.

Receiving medical data from at least one further device may, e.g., start before implantation and end before implantation, end right at the stage of implantation, or end after implantation. In the last example, there may be a stage and/or a phase during which the further device and the implanted medical device provide medical data to the system (simultaneously).

In some examples, similarly as before implantation, the method may include receiving medical data from at least one further device after the implant has been removed from the respective patient.

The method may, for example, further comprise sending a data request to the at least one further device at least in part based on the medical data from the at least one further device, the at least one first medical device, and/or the at least one second medical device. Sending such a data request may further increase the degree of automatization of the system, improve the efficiency of the system such that only those data that are actually required are being provided, for example, in response to the data request.

A data request may, for example, be sent based on a trigger as described herein, for example when one or more parameters of the medical data reach predetermined threshold values. The medical data requested as well as the medical data, based on which the data request may be sent, may originate from different medical devices or the same medical device. Different medical devices or the same medical device may be involved in different phases and/or stages of the therapy sequence, in this context. For example, the medical device in the trial phase may be a trial device while in any post-implantation phase the medical device may be an implanted medical device.

The above example may especially be relevant in stages before implantation of a medical device where specific data may be required to assess whether the patient is suitable and or qualified to receive an implantable medical device. This data may then be requested (automatically) from at least one further device based, e.g., on data from at least one other further device.

The method may, for example, further comprise one or more steps of validating the medical data and/or other data as described herein. Such validation of data may for example involve a clinician or an otherwise qualified health professional who may assess, for example, patient-reported data and/or device-reported data to ensure a solid basis for decisions on the patient treatment in terms of validated data. This may especially be beneficial in the beginning of the patient therapy sequence to ensure that the initial understanding of the patient on how to report data and/or the initial settings of the medical devices reporting medical data are correct.

The validation of the medical data may also occur in an automatic way, for example performed by a server of the system described herein.

In another example, the method may further comprise processing the medical data for a diagnostic identification of issues and/or for providing visualization data for visualization of the medical data.

This may allow cross-departmental or cross-company collaboration by providing a single point of status and notifications to responsible staff for upcoming or past-due events, critical patient care messages, long-term device status and health, and clinically relevant changes in patient health metrics. In particular, any user may thereby receive sufficiently processed medical data which may therefore be understandable without being an expert in the field in which the respective data were acquired. Hence, the issues and/or data visualization may be easily accessed by a user e.g., via access to a database as described herein.

In an example, the processing may be at least partly based on the stage of the patient. For example, depending on the stage of the patient, various issues may arise, and the data may be processed such as to exclude respective issues in a targeted manner. Similarly, stage-dependent reports may be desirable and such reports may be created automatically. This example yields the advantage that the existing framework of determining a stage of the patient in the patient treatment sequence may be further exploited for the automatization of the system and a more reliable, consistent but yet situation/stage-specific processing of the medical data. This may increase the overall efficiency and accuracy of the method and respective systems increasing the quality of care that may be provided to the patient.

For example, in the beginning of the patient treatment sequence, the data processing may comprise gathering data on basic patient information like medical issues, physiological and psychological examinations, patient insurance, etc. and validating these data. The data processing required during the trial phase may mostly be focused on assessing whether or not the trial medical device that the patient might be carrying during this phase has a sufficient effect on the patient health or not. In contrast, the data processing in the post-implantation phase may rather be focused on providing standardised report sheets for remote monitoring of the patient on a regular basis as described herein.

The method may further comprise forwarding the medical data for outputting to a user-interface, e.g. a user interface as described herein. This may for example be based at least in part on a medical data request described herein.

Such data collection methods enable global, country, region, and clinic-level assessments of performance, patient demographics, throughput, and outcomes, allowing continuous process feedback to improve standard of care and medical device offerings. This may further allow cross-departmental or cross-company collaboration by providing a single point access to provide status and notifications to responsible staff for upcoming or past-due events, critical patient care messages, long-term device status and health, and clinically relevant changes in patient health metrics.

The forwarding may for example be performed by any of the servers and/or layers described herein, e.g., a secure medical device server and/or a database.

In another example, the method may comprise pausing the method, based at least in part on the medical data indicating a pause condition, until receiving a continuation input. Pausing the method may improve the method from an economic standpoint reducing the amount of unnecessary data processing, patient involvement, and/or energy consumption which may also reduce the workload of the involved users.

The pause condition may for example be fulfilled when authorization is required, or a detected issue is to be rectified. When such a pausing condition is met, the method may be paused in different ways: the automatic determination of the stage based on new incoming medical data may be paused while new medical data keep coming. Alternatively, a full pausing mode may be used, wherein e.g., the medical device or further devices, may be switched off until a continuation input is received. The continuation input may for example be a message or a notification that authorization was successful and/or a detected issue was rectified.

In some examples, the method may comprise terminating the method, based at least in part on the medical data indicating a termination condition. Identifying termination conditions correctly and acting thereon by terminating said method poses the advantage that no further unnecessary effort is made for progressing patients along further stages when it is already known that they may for example not be suited to receive an implant which may be one of the goals of the rolled-out therapy sequence.

Typical termination conditions may for example comprise identifying physiological or psychological reasons why the patient may not be suitable to receive an implant, regulatory criteria (e.g., when the patient has no insurance), etc.

In an example, defining the patient stage may further comprise accessing one or more predetermined criteria of the patient stage, and preferably further comprising a determining whether the one or more predetermined criteria are met, at least in part based on the medical data. This may allow a reliable tracking of the patient stage.

For example, the predetermined criteria of the upcoming stage that the patient might progress to are accessed to check whether these criteria are met. The predetermined criteria may, for example, comprise parameter ranges and the respective criterion may be met when one parameter of the medical data lies within said parameter range. In this case, the patient may then progress to the next/upcoming stage.

The repeating of the different steps of the method may, e.g., occur regularly according to a predetermined repetition schedule. Following a predetermined repetition schedule and thereby regularly checking whether the patient may have progressed to the next stage of the therapy sequence ensures a fast progress of the patient and avoids unnecessary waiting times.

The repetition schedule may for example be set up such that the steps of the method are repeated periodically (hourly, daily, weekly, monthly) and/or at predetermined times (e.g., scheduled by clinicians and/or linked to special events like doctor's appointments etc.). Different steps named herein may be repeated at different repetition schedules or at the same repetition schedule. For example, continuous automatic data monitoring may be performed daily while reassessing trigger thresholds stored in the system may be reviewed on a monthly basis.

The method may, e.g., further comprise decrypting the medical data from the at least one first and/or second medical device, preferably into a file structure with key-value pairs, more preferably in a human-readable language. This may increase the accessibility of the medical data and increase the user-friendliness of the method and system. The decrypted data may then, e.g., be in a JSON-file format and/or any other similar file format.

The method may, e.g., further comprise on-demand reprogramming of at least one of the first and/or second medical devices; and determining the patient stage based on the reprogramming of the at least one medical device.

Generally, the medical data may comprise also data on the programming state of the medical device. Therefore, reprogramming itself may result in changing the conditions such that the patient may progress to the next stage of the therapy sequence. Additionally, or alternatively the system may recognise that a reprogramming is necessary for the patient to progress and such reprogramming may be performed at least in part automatically. This may improve the quality of care provided to the patient and the efficiency and accuracy at which the respective medical device may be operating. Referring to the current stage of the patient in programming the medical device may further help to make better, number-based decisions in how to program the medical device.

The programming may be performed on-demand, not necessarily as a part of the repetition schedule and/or any other regular schedule.

In some examples, the method may further comprise sending at least one reprogramming instruction to at least one of the first and/or second medical devices to switch from a first program to a second program. Subsequently, the determining of the patient stage may be based at least in part on the second program.

Another example of the second aspect of the invention provides a computer program comprising instructions to execute the steps of the method described herein.

Another example of the second aspect of the invention provides a system for automated patient treatment sequencing, the system comprising means for receiving medical data from at least one first medical device, means for defining a patient stage within a predetermined patient treatment sequence, based at least in part on the medical data, means for receiving further medical data from the at least one first medical device and/or at least one second medical device, and means for defining the patient stage within the predetermined patient treatment sequence, based at least in part on the further medical data.

The respective computer programme and system may provide the same advantages described herein in reference to the method.

A third aspect of the invention is described in the following:

According to the third aspect of the invention, a method for automated patient assistance is provided. The method comprises receiving medical data from an external device associated with a patient, defining a first patient stage within a predetermined patient treatment sequence, based at least in part on the medical data from the external device. It may further comprise receiving medical data from an implant of the patient and defining a second patient stage within the predetermined patient treatment sequence, based at least in part on the medical data from the implant. Notably, the medical data from the external device may be received before implantation of the implant. The method may further comprise providing educational material to the patient, based at least in part on the patient stage.

This may, e.g., improve the efficiency of the workflow as well-informed patients may be more likely to actively participate when requested and are less prone to use any of the devices involved in the process incorrectly. Further, this may increase user satisfaction and comfort. All this may occur remotely, instead of during in-person appointments at the doctor's office to inform patients, which saves time and costs.

The patient stage may be defined, e.g., as described herein with respect to the second aspect.

The method may, e.g., further comprise registering the patient; and defining the patient treatment sequence, comprising a plurality of stages, based at least in part on the registering.

This may provide a robust and automatic tool to determine a suitable patient treatment sequence which may comprise the stages the patient may progress through, one after another. This may provide an advantageous framework for determining the current stage of the patient based, e.g., at least partly on the medical data and/or checking whether they meet predetermined criteria of the upcoming stage that the patient may progress to.

A further method for automated patient assistance may be provided. The method may comprise registering a patient, defining a patient treatment sequence, comprising a plurality of stages, based at least in part on the registering. It may further comprise receiving medical data associated with the patient and defining a patient stage within the patient treatment sequence, based at least in part on the medical data. Further, it may comprise providing educational material to the patient, based at least partly on the patient stage.

This may, e.g., increase the degree of automatization of the workflow by setting up all the stages for the patient to go through one after another from the beginning of the process, directly based on the registration process. This may increase plannability and allow to far-sightedly make preparations for upcoming steps and/or stages which may at this point already be known to occur at a later point of the patient treatment sequence.

This method may, e.g., result in providing the patient with information via, e.g., a patient app. Such app may provide an information access system which scales information visibility and consent according to a candidate patient's progress/journey along the care continuum/patient treatment sequence. Candidate patients can, for example, initially easily access an app download via a digital link or QR code where they enter, e.g., demographic information and/or other information that may at least partly be the basis for determining the patient treatment sequence. The patient may then have access to basic information in response (for example, about pain management and local physicians). The digital link or QR code may e.g., be sufficient to provide the information required to define the patient treatment sequence including the stages the patient may progress through, one after another. Such QR code or link may for example be provided to the patient by the attending health professional who may assist the patient along the patient treatment sequence, for example aiming at providing the patient with a suitable implant for ongoing remote monitoring of the patient. The digital link or QR code may for example be device-, country-, region-, health professional-, and/or patient-specific. Thereby the advantages of providing an individualized, but at the same time robust, patient treatment sequence are combined, e.g., by choosing from an existing library of predetermined patient treatment sequences comprising corresponding stages. Such library of sequences may for example be stored in one of the databases and/or secure medical device servers of a system as described herein. The suggested patient treatment sequence may, for example, be further accepted and/or adjusted by the attending health professional.

The basic patient information provided upon registration (e.g., via a user interface to a database as outlined herein) may be transmitted to a central server (e.g. a secure medical device server) for continuity at a later date, should the candidate become a patient. At a later stage, following physician contact, the candidate may be upgraded to “patient” status via a validation process where the candidate's status as a patient (or a more specific corresponding stage or phase) is confirmed. At this point, the downloaded application may allow access to deeper information about their future continuum of care (for example, SCS trial, then subsequently to possibly permanent implant) and may provide further questionnaires to begin collecting more specific data about the care pathway the patient is continuing along.

Later, as the patient completes further stages of care, the questions and information provided are more and more specific to their condition and treatment options. In addition, access to specific support groups may be offered at certain stages of the patient treatment sequence.

For example, the method may further comprise receiving medical data from an external device associated with the patient and defining a first patient stage within the predetermined patient treatment sequence, based at least in part on the medical data from the external device. It may further comprise receiving medical data from an implant of the patient and defining a second patient stage within the predetermined patient treatment sequence, based at least in part on the medical data from the implant. The medical data from the external device may be received before implantation of the implant.

Further, the registering may, for example, comprise a regional identification and/or a regulatory identification associated with the patient, wherein defining the patient treatment sequence is based at least in part on the regional identification and/or the regulatory identification.

This may have an advantageous effect, as regional and regulatory aspects to consider may be known from the beginning and may be considered in determining the stages of the patient treatment sequence. Such regional and regulatory aspects may, for example, relate to health standards in the respective country and/or region, the accessibility of health institutions, medical standards to fulfil, etc.

For example, the educational material may comprise at least one of: information about medical device (e.g., implant) and/or external device operation, information about medical device (implant) and/or external device settings, contact information of the medical device (implant) and/or external device manufacturer and/or involved clinicians, support information, troubleshooting information, and links to access any of this information.

Thereby, the patient may, for example, receive sufficient information to be well-informed about their current stage as well as potential upcoming stages of the patient treatment sequence. This may increase patient comfort and satisfaction with the overall process. Less patients may be lost along the path of setting them up to receive an implant, improving the efficiency of the process. Furthermore, such well-informed patients may be less prone to misunderstand any of the instructions given to them and/or to use any of the medical devices incorrectly.

Information about medical device settings etc. may, for example, comprise manuals for the medical devices associated with the patient.

In an example, the educational material may comprise at least one of: a video, a text, an audio information, an image, a web-link, and a combination thereof.

Providing the patient with a variety of different information formats may increase the chances of the patient accepting the offered content. Further, disabled patients may also benefit from this. For example, a blind patient may still be able to receive all relevant information when these are provided as audio information.

The patient treatment sequence may, e.g., comprise at least three patient stages, wherein at least partly different educational material is provided in each of the at least three stages.

This allows for a sufficiently fine breakdown of the patient treatment sequence into individual stages during which any of the functionalities described herein may be performed based at least partly on the current stage of the patient and/or upcoming and/or past stage(s) of the patient. This may increase the suitability of the functions performed on each stage, increasing the overall quality of care that may be provided to the patient.

The method may, e.g., further comprise outputting a notification on the patient stage and/or on the educational material for the patient.

Such notification may for example be (or lead to a) push notification that the patient receives via a patient app on their mobile phone. This may for example be the same application that also provides the educational material to the patient. The notification may, for example, inform the patient that they progressed to a new stage and/or that new educational material is available for them. This may advantageously involve the patient and remind them to use the application and/or the educational material offered to them, which may increase the success chances of the overall treatment as described herein. The notification may also remind the patient to access (e.g., view) educational material when they have not done so for a predetermined time after the educational material became accessible to them, e.g., after a day, a week, a month, etc.

The method may, e.g., further comprise forwarding the educational material for outputting to a user-interface. For example, the educational material may be stored in a database of the system as described herein and may be forwarded within the system to be outputted by the user-interface. The user-interface may, for example, be the user-interface of an application run by the patient's mobile phone, e.g., in a web-based application. When the educational material is forwarded as described herein, it does not necessarily have to be stored on the mobile phone or any other device used for outputting the educational material where there might be storage restrictions, for example due to space restrictions and/or misuse of the patient.

The forwarding of the educational material may, for example, comprise forwarding the educational material in a preferred hierarchy, suggesting an order to the patient in which to view the educational material.

This may further increase user satisfaction and increase the chances that the system described herein is used properly by the patient. In an example, the most important educational material may always be forwarded such that it is presented to the patient, for example, as the first item of a list of educational information. For example, the preferred hierarchy may ensure that the most relevant educational information is always shown in the first position of a list shown by, for example, an online portal or the patient app on the patient's mobile phone.

The method may, for example, further comprise receiving a data request via the user-interface. Forwarding the educational material may be at least partly based on the data request.

In this example, the educational material may, for example, be provided to the user interface on demand and only when requested by the patient, for example by clicking on a field of the patient app interface, e.g., to select the given educational content. In the example of the patient app, the app interface may, for example, show a short preview of the educational material. when clicking on that preview, the patient app may send a data request requesting the associated educational material. The system as described herein may then trigger a forwarding of the respective educational material from the database where the educational material may be stored to the user-interface. In other examples, the system may have stored the educational material and forward it to the database for outputting to the patient.

Further, a computer program is provided that comprises instructions to execute the steps of the method as described herein.

Further, a first system for automated patient assistance is provided. Said system comprises: means for receiving medical data from an external device associated with a patient; means for defining a first patient stage within a predetermined patient treatment sequence, based at least in part on the medical data from the external device; means for receiving medical data from an implant of the patient; means for defining a second patient stage within the predetermined patient treatment sequence, based at least in part on the medical data from the implant; and means for providing educational material to the patient, based at least in part on the patient stage.

Further, a second system for automated patient assistance is provided. Said second system comprises: means for registering a patient; means for defining a patient treatment sequence, comprising a plurality of stages, based at least in part on the registering; means for receiving medical data associated with the patient; means for defining a patient stage within the patient treatment sequence, based at least in part on the medical data; and means for providing educational material to the patient, based at least partly on the patient stage.

These two systems may pose all the advantages described here in reference to the steps of the method: The method, preferably using a patient app which is freely accessible to all users of a unified application distribution channel, may initially collect basic demographic information about the user, and allows the user to scan a QR code at a physician office to associate themselves with that office's care team. In this way, a candidate's status may be tracked, proper information may be provided at each stage, and a single application and central system may be used to provide customized information to patients throughout their patient treatment sequence. Advantages of the invention may include: a more convenient registration process for the patient through QR code scanning; more informed patients throughout the entire patient treatment sequence, from prospective patient to receiving active therapy; accessibility of the patient app to patients, with the relevant information and features provided to them at each stage of their journey; an improved patient experience; unified milestones for consistency of feedback collection; simplified coordination of patient activities with their registered local clinic, etc. These advantages may apply to all the above methods, computer programs, and/or systems.

The methods herein may be computer-implemented methods, and a computer program may be provided with instructions according to the steps of the methods. Further, any functionality described herein in reference to the system may be implemented as steps of a method and/or as instructions of a respective computer program, and vice versa.

Computer programs may be stored in any storage device, and the various method steps may be implemented in software or hardware or a combination thereof.

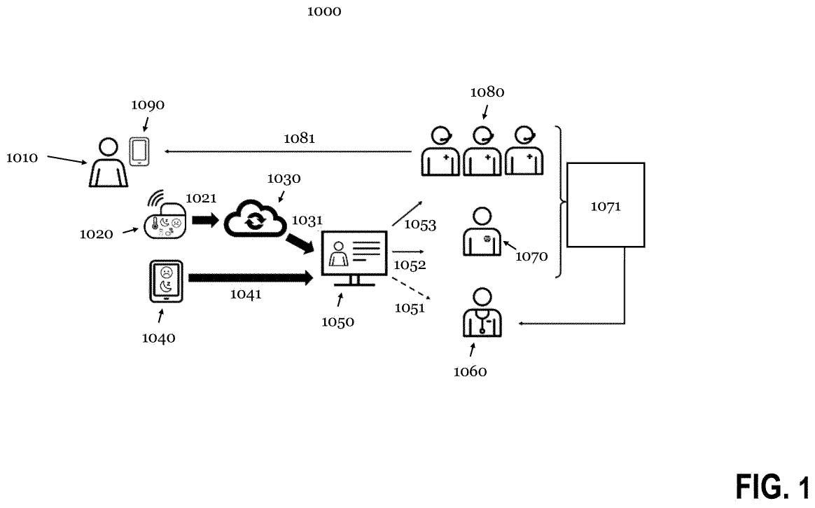

FIG. 1 shows a schematic representation of a communication infrastructure for medical device data allocation to various recipients and defining patient stages on a high level.

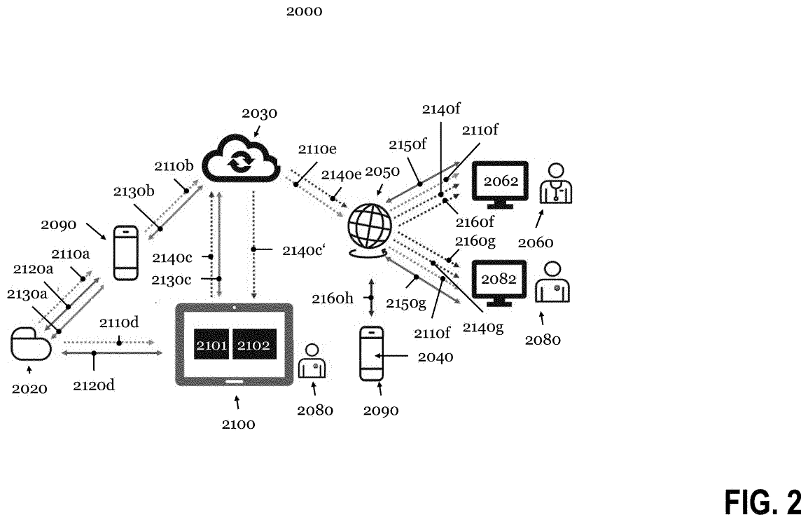

FIG. 2 shows a schematic representation of a communication infrastructure for medical device data allocation to various recipients and defining patient stages on a more detailed level than FIG. 1.

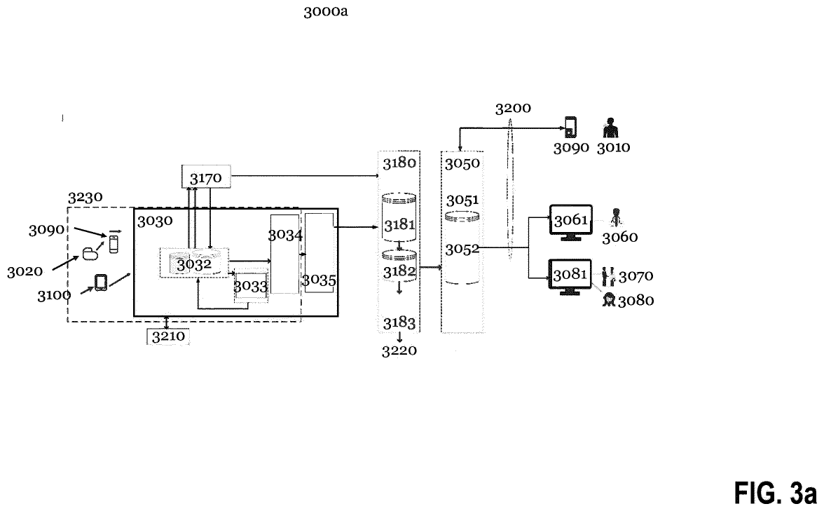

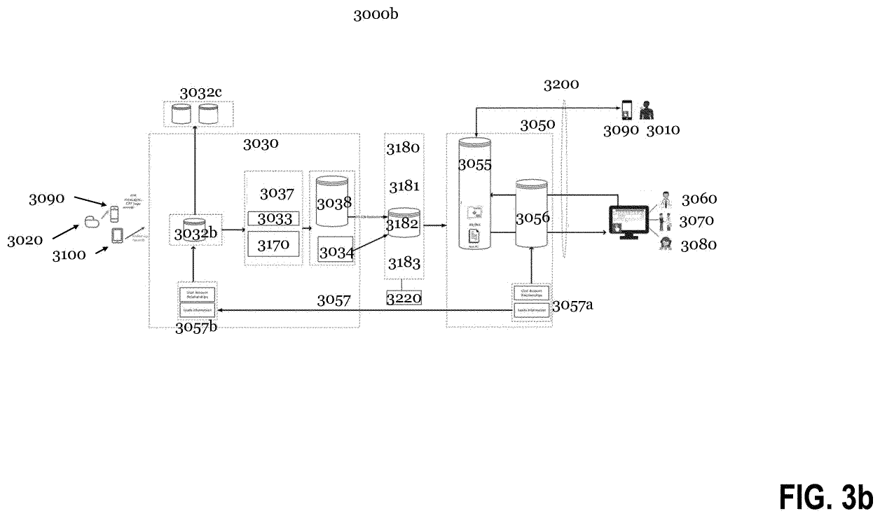

FIG. 3a shows a schematic representation of a communication infrastructure for medical device data allocation to various recipients and defining patient stages with a focus on the intermediate layers managing data transfer and processing between the medial device and the recipients.

FIG. 3b shows a schematic representation of a different communication infrastructure for medical device data allocation to various recipients and defining patient stages with a focus on the intermediate layers managing data transfer and processing between the medial device and the recipients.

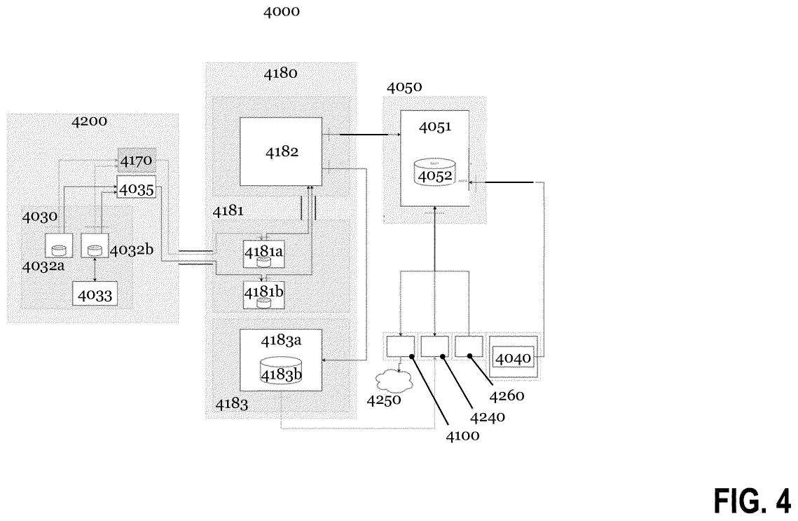

FIG. 4 shows a schematic representation of a part of the communication infrastructure for medical device data allocation to various recipients and defining patient stages with a focus on the architecture and internal data transfer of the intermediate layers managing data transfer and processing.

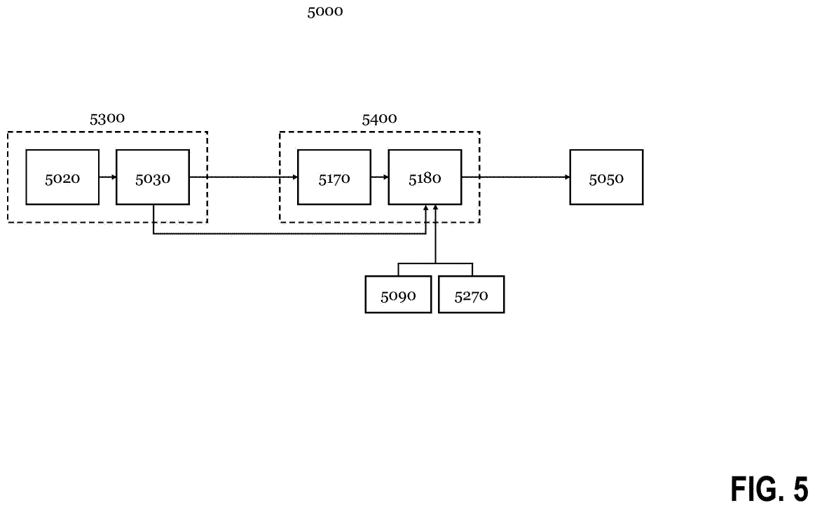

FIG. 5 shows an abstracted schematic representation of the communication infrastructure for medical device data allocation to a user interface and for defining patient stages of FIGS. 1-4 highlighting an exemplary grouping of involved components into two blocks.

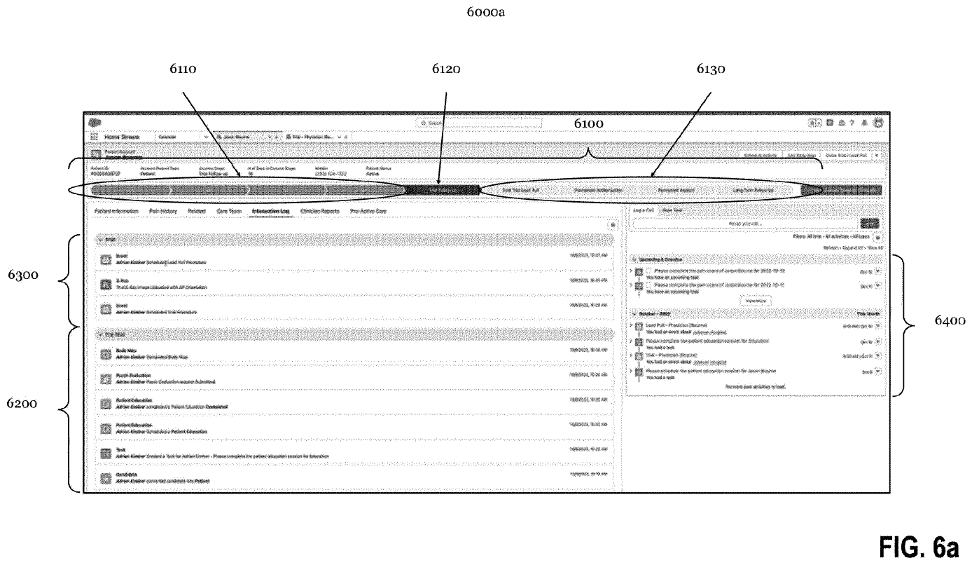

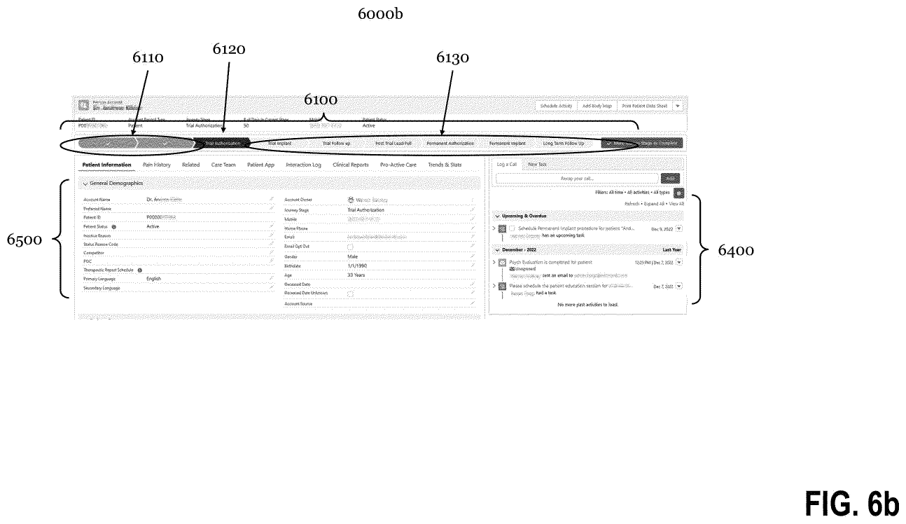

FIG. 6a shows an exemplary graphical user-interface related to the portal backend of an exemplary system.

FIG. 6b shows another version of an exemplary graphical user-interface related to a portal backend of an exemplary system.

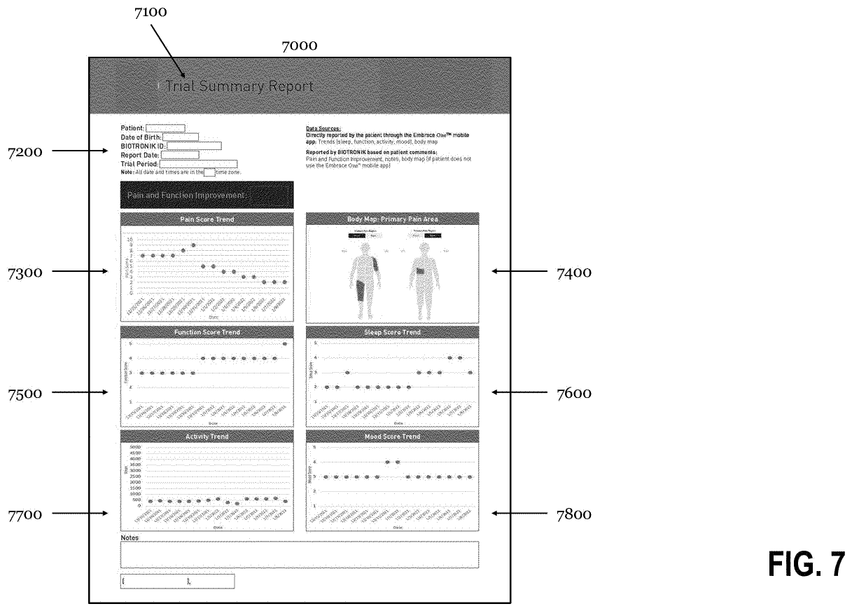

FIG. 7 shows an exemplary trial summary report.

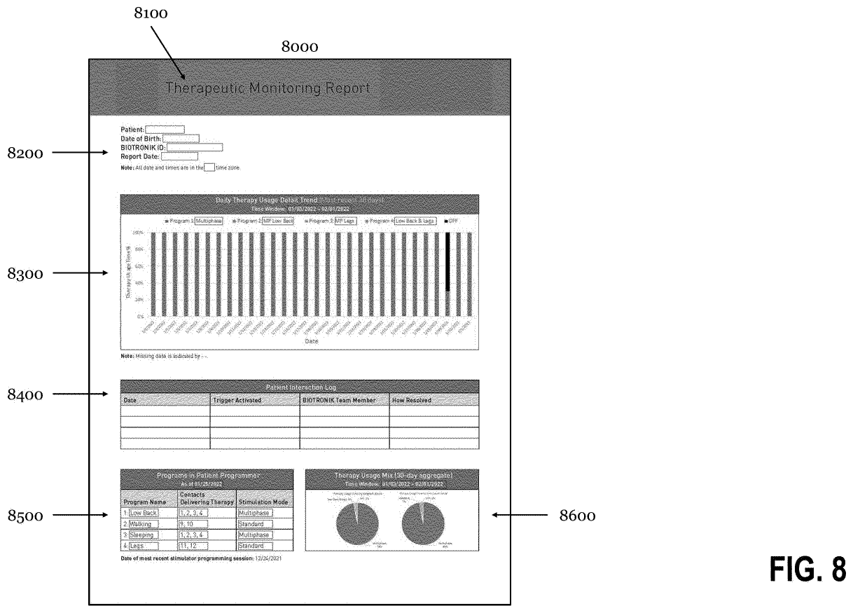

FIG. 8 shows an exemplary therapeutic monitoring report.

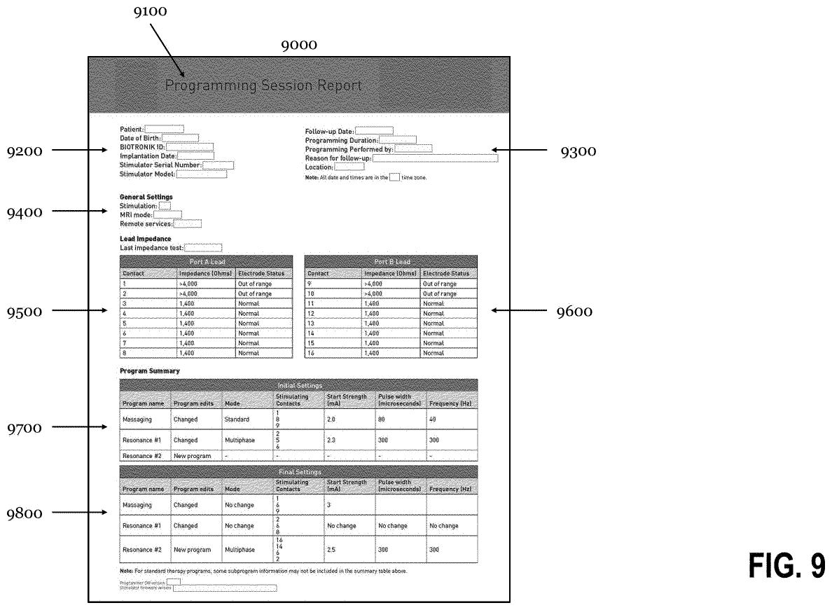

FIG. 9 shows an exemplary programming session report.

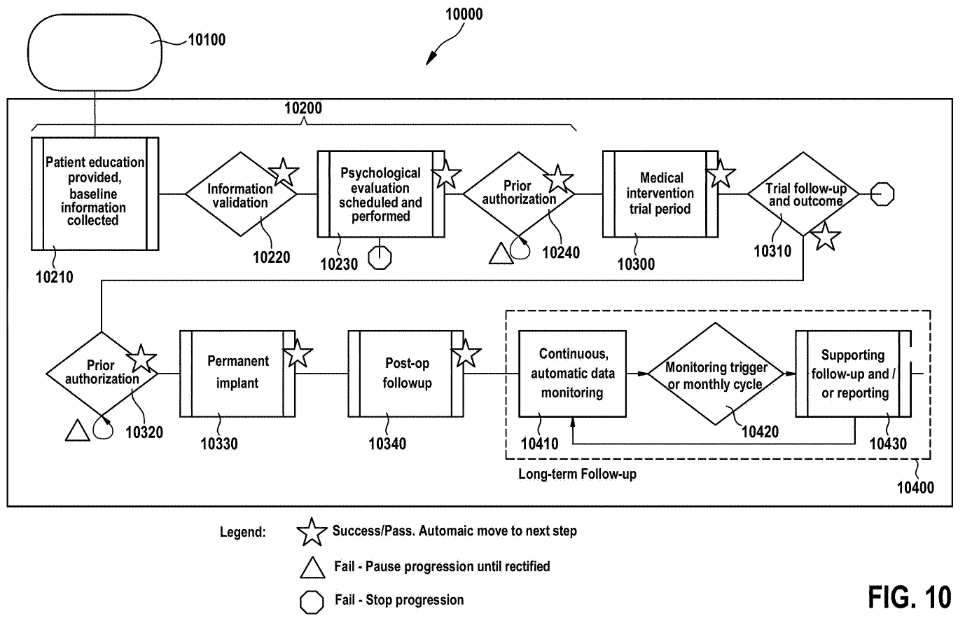

FIG. 10 shows a flow diagram of a preferred embodiment of a system for defining patient stages, e.g., a patient triage and automatic journey sequencer.

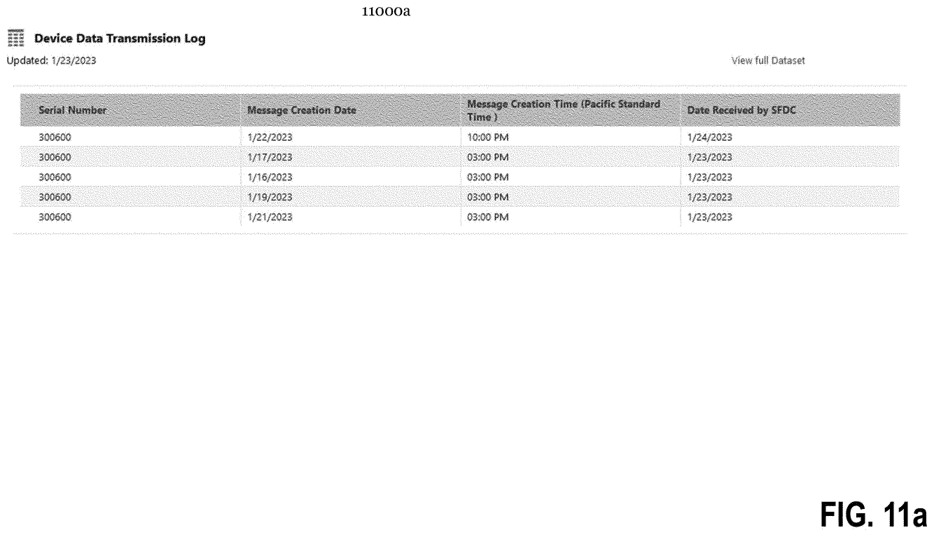

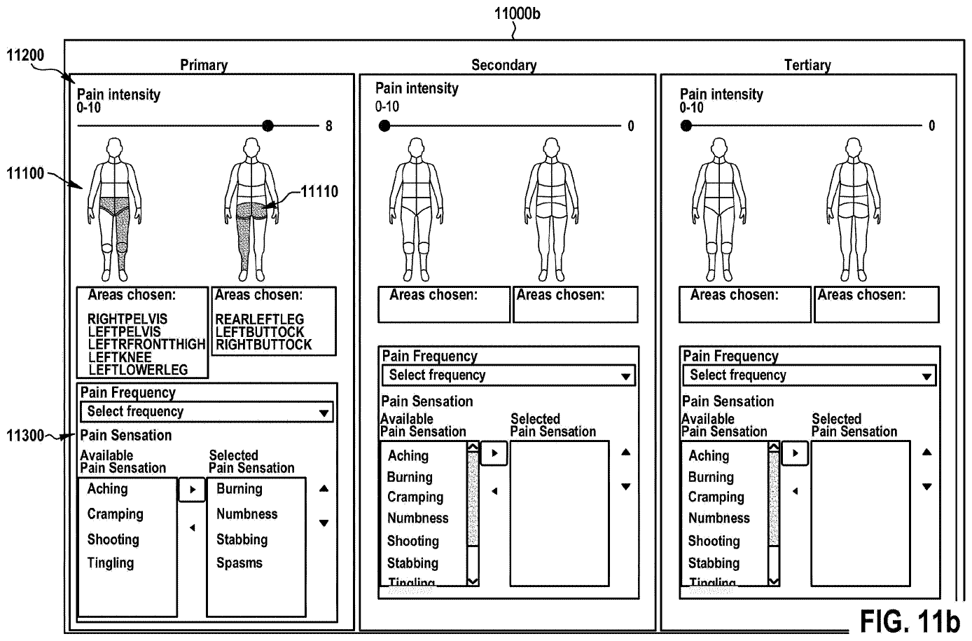

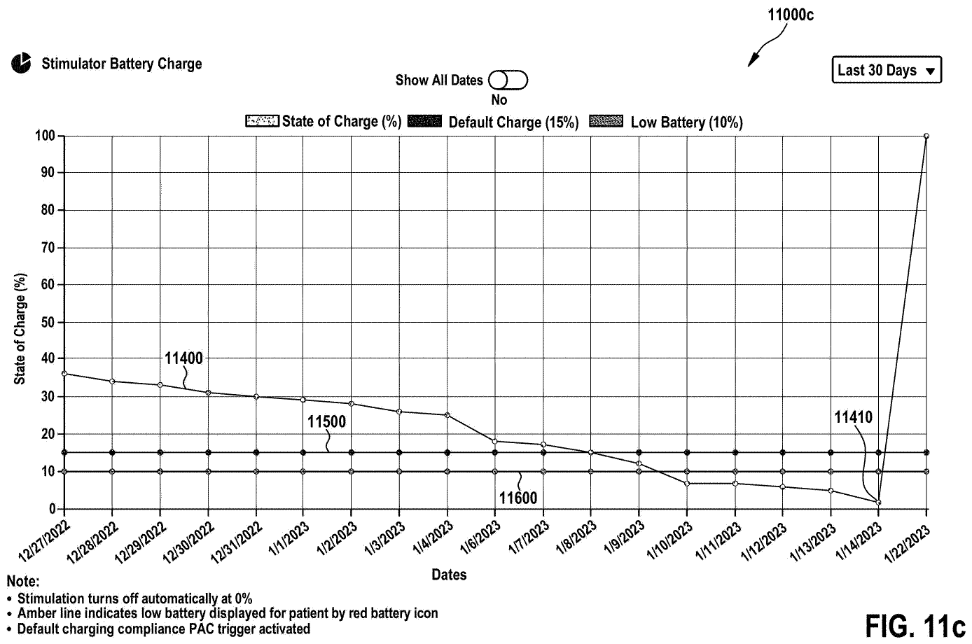

FIG. 11a shows an exemplary device data transmission log.

FIG. 11b shows a user interface for data input for the identification of primary pain areas.

FIG. 11c shows a long-term follow-up display of a medical device battery charge and a recent recharge.

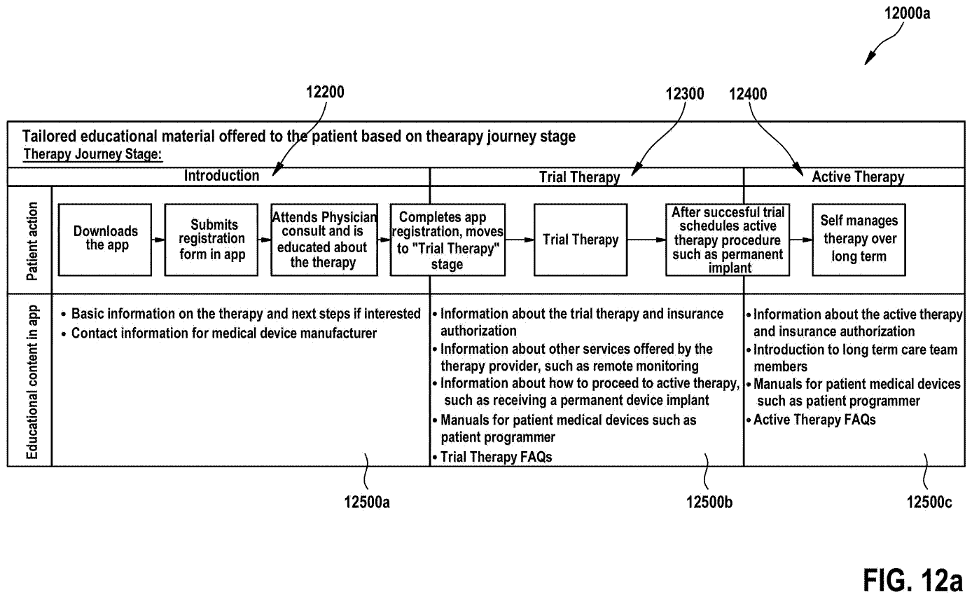

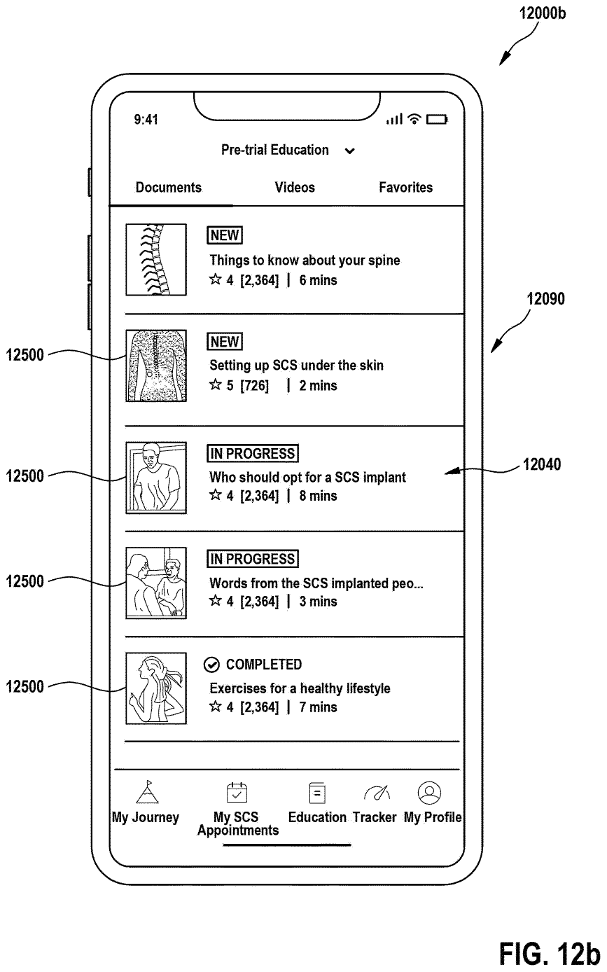

FIG. 12a shows a flow diagram linking the current patient state in the patient journey with educational material provided to the patient according to the patient state.

FIG. 12b shows a graphical user-interface of an exemplary patient app run by a patient remote showing selected educational material based at least in part on the patient stage in the patient journey.

The description of preferred embodiments provided here focuses on a unique solution for implantable and external Spinal Cord Stimulation (SCS) devices and systems. However, this same approach could be used in other remotely monitored medical device systems, in particular implants, e.g., cardiac rhythm management devices, etc.

FIG. 1 shows a schematic representation of a communication infrastructure 1000 for medical device data allocation to various recipients 1010, 1060, 1070, 1080 and defining patient stages on a high level to illustrate the general working principle of an exemplary embodiment of the present invention.

A patient 1010 may connect to the system 1000 via a patient remote 1090, e.g., a mobile phone, running an application 1040 allowing the patient 1010 to insert data and/or view data in system 1000. A medical device 1020 may be associated with the patient 1010 and transmit medical device data 1021, e.g., in regular/periodic/daily (automatic) transmission, to a secure medical device server 1030. The patient application 1040 may be used by the patient 1010 to input further data, e.g., patient manually reported data, and the application 1040 may, in some embodiments, automatically transmit at least a part of the received data 1041 to the secure medical device server 1030 (not shown in FIG. 1). The data 1041 may be transmitted in automatic, near real time data transmission. The secure medical device server 1030 may be configured to receive medical data 1021 from one or more medical devices 1020, e.g., in a controlled/programmable/on-demand or automatic way. The secure medical device server 1030 may act as a central node that may collect data 1031 and 1041. It may collect data from more than one medical device 1020 and more than one patient application 1040.

After additional processing by the secure medical device server 1030 and/or by further components (not shown), the data 1031 may be transmitted in an at least partly modified form to a database and/or portal backend 1050.

In the example shown in FIG. 1, data 1041 is not routed through the secure medical device server 1030, but it may be transmitted to the database and/or portal backend 1050 via other paths.

Via, e.g., a web-based user interface, users may access remote monitoring portal 1050 such that the data 1031 and 1041 may at least partly be output in their original or in a processed form to any authorized recipient.

Users may provide proof of authorization, e.g., by logging into the remote monitoring portal 1050 via a username and password. As shown in FIG. 1, the remote monitoring portal 1050 may transmit data 1051 to a healthcare provider 1060, e.g., in the form of portable document format (PDF) reports. It may further transmit data 1052 and 1053, respectively, to a local technician 1070 (e.g., a device specialist at a clinic and/or device specialist of the device manufacturer) and/or a remote care team 1080 (e.g., device specialists of the device manufacturer and/or device specialists at a clinic). The local technician 1070 and/or the remote care team 1080 may provide a report 1071, e.g., on early issue detection, potential (fast) issue resolution, etc., to the healthcare provider 1060, e.g., via e-mail, text, or any other means of electronic health record systems, etc. The remote care team 1080 of FIG. 1 may also provide remote programming instructions 1081, when needed, to the patient remote 1090 which may act as relay device for remote medical device 1020 (re-)programming. This may alternatively or additionally also be done by the local technician 1070.

In a simple example, the portal 1050 itself and the associated microservices software functions are non-medical device functions and are therefore regulated as such, although they are provided with patient medical data via a medical device connectivity system (MDCS), e.g., including secure medical device server 1030. The software does not control nor alter any parameters on the therapy devices. The portal 1050 in this embodiment makes no diagnostic or treatment suggestions. It simply reports the information that is collected. The data can be viewed, e.g., in Salesforce.com or other browser-based application. The shown data may comprise, for example:
a) A follow-up history on all interactions with remote care team 1080 and follow-up programming reports for both, in-person and remote follow-ups 1081.
b) Patient monthly device reports on, e.g., when the medical device 1020 is a SCS device, stimulation usage, therapy adjustments, and lead impedance, and/or information on cost reimbursements.
c) Proactive care and outcome trends comprising population and/or individual patient 1010 statistics, e.g., on utilization of proactive care, outcomes, and/or other population information.

Overall, FIG. 1 illustrates a general concept of how one (or more) patients 1010, one (or more) healthcare providers 1060, one (or more) local technicians 1070, and/or one (or more) remote care teams 1080 may access data from and/or provide data to a medical device 1020 of a patient 1010 and/or to the patient 1010. The portal 1050, e.g., via a (portal health cloud) app, e.g., run on the patient's phone 1090 may display patient reported outcomes from the patient app 1040. Data from the patient app 1040 is automatically and remotely transmitted from the patient's mobile phone 1090. Additionally, patient app data 1041 may be stored in a business intelligence layer (not shown) of the system 1000, as described herein. As an alternative, patients can also report directly (verbally) to the care team 1080 and the information can be manually logged by the care team 1080 into the portal 1050. Additional patients' data may come via responses to clinically validated surveys (e.g., PROMIS-29 and Oswestry Disability Index (ODI)) which are captured and displayed in the portal 1050. Completed survey data may, e.g., be stored in the business intelligence layer (not shown), as described herein. Lastly, patient satisfaction surveys completed via the patient app 1040 may be displayed in the portal 1050.

In the exemplary system 1000 of FIG. 1, various components may decrypt and/or process data in other ways and/or determine a patient stage within a predetermined patient treatment sequence, as described herein. The components that may execute these functions comprise, e.g., the secure medical device server 1030 (e.g., a component thereof), the portal backend 1050, and/or any component via which the data 1031 may be transmitted between the medical device server 1030 and the portal backend 1050. The portal backend 1050 may, for example, comprise the second database, described herein.

The portal backend 1050 may thus provide a universal interface for users that may be easy to use. This may be enabled by receiving at least partly decrypted medical data from a processing system that receives encrypted medical data from the secure medical device server 1030 and at least partly decrypts it, and thus serves as an interface between the medical and the non-medical sides of the system.

The following information can be displayed on the portal (health cloud) 1050 for each patient 1010: patient reported outcomes (patient functional goals, patient's perceived progress to functional goal, statistical outcomes summary of the parameters below including the most recent reported value with timestamp, and change to previous week or month (e.g., pain score, sleep quality, mood score, activity, function, medications, and/or daily compliance)), patient satisfaction rating, daily trend for patient-reported pain score, sleep quality, mood, activity, function, and/or medications over a minimum of, e.g., 30 days in history, pain region history including pain region, sensation, and intensity, image of baseline and most recent pain body map, compliance in reporting outcomes in the Patient App through the following metrics (e.g., date of most recent update of pain score, date of most recent update of body pain map, and/or date of most recent update of functional goals). Device data received from the business intelligence layer (not shown) may be displayed and trended in the portal (health cloud) 1050 for use in remote monitoring processes. It should be noted that the named types of displayed data are to be considered as examples. Other types of displayed data are possible and incorporated in the scope of the present invention. The care team 1080 may proactively monitor the device data 1021 through the portal 1050 to determine if there is a reason to believe the patient 1010 might need further assistance to ensure compliance with proper usage of the system. For this purpose, the user 1060, 1070, 1080 may view a device data report 1051, 1052, 1053, which identifies patients 1010 who meet certain pre-defined criteria, with a column indicating the metrics which have been met based on criteria as shown in Table 1. These metrics may be updated automatically by the system 1000. The care team may also reach out to patients 1010 proactively based on data 1053 viewed in the portal 1050.

TABLE 1

| Metric | Objective | Metric Criteria |
|---|---|---|
| Active Electrode | Identify patients with | Contact impedance in |
| “Out of Range” | stimulating contacts | defined program <200 |
| | (currently or in most | Ω or >4000 Ω |
| | recent program) outside | |
| | of a specified range of | |
| | impedance values. | |
| Low Device Usage | Identify patients using | Multiphase % |
| | therapy for a lower | time <100% |
| | duration than a specified | OR % Stimulation |
| | duration of time per day. | OFF = 100% in most |
| | | recent day reported |
| Sub (or | Identify patients who are | Average |
| Over)-therapeutic | operating stimulation | amplitude <0.4 mA |
| Amplitude | with amplitude outside of | OR Average |
| Monitoring | a defined range of values. | amplitude <0.8 mA |
| | | for tonic therapy |
| Charging | Identify patients with a | Battery Sate of |
| Compliance | stimulator battery charge | Charge (SoC) <10% |
| | level below a specified | |
| | value. | |
| Data Transmission | Detect gaps in data | Time since last |
| | transmission from the | transmission >48 |
| | patient's SCS system to | hours |
| | the remote monitoring | |
| | interface. | |

In this manner the medical device, in the following, e.g., an SCS, experience may be improved for patients 1010 by ensuring compliance with therapy, monitoring for potential changes in the SCS system 1000, and identifying areas for patient education to ensure patients 1010 get the most out of their therapy. This service may be delivered through the following actions: (i) Evaluation of the device usage 1020 and integrity to ensure patient compliance with their prescribed therapy; (ii) Evaluation of device usage behavior to provide education to patients 1010 on how to optimize the use of their device 1020.

Using device data, the portal 1050 may automatically identify patients 1010 who meet metric criteria/triggers for inclusion in a device data report. The device data report can for instance be triggered on-demand or automatically produced on a regular basis (e.g. daily). This serves as an initial filter of patients to be prioritized for further assessment. The care team 1080 will first review the data in detail, and may then reach out to the patient 1010, gather more information to assess the patient situation, and, if appropriate, take action to assist the patient 1010 through education, appointment coordination, or device reprogramming. In addition to the device data report, the care team 1080 may also review additional device data statistics and trends to determine necessary patient outreach. After a patient has been on the device data report list, or any time a user 1060, 1070, 1080 may reach out to the patient 1010, the care team 1080 may then update the patient status for ongoing patient management.

The care team 1080 will have access to device diagnostic data trends and statistics. This data is supplemental information for the care team 1080 to assess the patient's usage of the device 1020. Device trends and statistics allow the care team 1080 to perform, e.g., three main objectives: (i) Evaluate technical lead impedance trends to understand when an electrode failure occurs; (ii) Evaluate patient therapy usage (e.g. including stimulation amplitude and program selection) to understand the patient's 1010 normal use patterns; and/or (iii) Evaluate the device battery levels and charging history to assist with troubleshooting and educating the patient 1010 on potential charging usability concerns. When performing patient outreach, the care team 1080 may reference device technical statistics as an aid in assessing the patient's device usage. In addition, the care team 1080 may review device data 1021 periodically for the purposes of ongoing technical monitoring. Finally, when a patient 1010 or physician 1060 calls with an inquiry, the care team 1080 may refer to the device technical trends and statistics as part of the troubleshooting process with the patient 1010.

The portal (health cloud) 1050 may also allow the care team 1050 to export the patient therapy response and device data 1021 into a file for statistical analysis at a patient population level or by clinic or following physician. These data may be deidentified in compliance with, e.g., Health Insurance Portability and Accountability Act (HIPAA) regulations.

Primary workflows of the system 1000 described herein may comprise the following:
1) Cross validation of patient data with assigned stimulator serial number: Messages sent from the secure medical device server 1030 may include: a patient ID and a stimulator serial number at the time the messages were created. Additionally, these messages may contain patient identifying information, including: a patient name (first and last) and a date of birth. If the message patient ID exists in the portal/database 1050, the portal/database 1050 validates that the incoming data is properly allocated to a given patient profile by comparing the stimulator serial number in the stored secure medical device server messages 1031 to the stimulator serial number assigned to the patient profile at the time of the message creation. If stimulator serial number and date matches, data transmission is successful, and data is displayed to user 1060, 1070, 1080. If the stimulator serial number does not match an expected number or value, data is discarded. It is expected that subsequently the user 1060, 1070, 1080 will notice data is not being displayed as the system will detect the data transmission issue through the “data transmission” proactive care trigger. If the patient ID does not exist in the portal/database 1050 database, then secure medical device server data 1031 are not displayed in portal/database 1050 and is lost/discarded. The user 1060, 1070, 1080 will notice data is not being displayed. Users 1060, 1070, 1080 may update the patient ID in the clinician programmer to match patient ID in the portal/database 1050 if necessary.
2) Proactive Care: The remote care team 1080 may monitor patients 1010 through the portal 1050 to proactively reach out to patients and gather information on the patient's situation if there is reason to believe the patient 1010 might need assistance. Proactive care aims to improve the SCS experience for patients 1010 by ensuring compliance with therapy, monitoring for potential changes in the SCS system 1000 and identifying areas for patient education to ensure patients get the most out of their therapy. Proactive care may be achieved through two critical pathways: (i) Evaluation of device usage and integrity to ensure patient compliance with their prescribed therapy, and (ii) evaluation of device usage behavior to provide education to patients 1010 on how to optimize the use of their device 1020. There may be five system-generated “proactive care triggers” which flag patients 1010 for remote care team 1080 evaluation. The proactive care triggers may comprise the metric criteria of Table 1. The following tasks may be performed as part of this proactive care model: (i) The user 1060, 1070, 1080 may generate a “Patient Triage” portal report which identifies patients 1010 meeting proactive care triggers among patients assigned to user-specified long term care physicians. This report may serve as an initial filter of patients who may benefit from outreach. (ii) The user 1060, 1070, 1080 may reach out to the patient 1010, gather more information to assess the patient situation, and, if appropriate, take action to assist the patient 1010. As part of this process, the user may review additional trends and statistics in the portal 1050 for further assessment of the patient's situation. (iii) Finally, the user 1060, 1070, 1080 may manually update the patient stage in the system for ongoing patient management. The interface 1050 allows the user 1060, 1070, 1080 to set a series of tasks to remind the user to continuously follow up with the patient as needed. It also allows patient 1010 to be temporarily flagged for ‘do not contact unless medically urgent’ for times when patient 1010 does not wish to be contacted (i.e., on vacation or a death in the family).
3) Trends & Statistics: Users may have access to viewing device diagnostic data trends and statistics (whether or not patient has met proactive care triggers). This data is supplemental information for the user to assess the patient's situation. Device diagnostic trends and statistics allow the user to perform three main objectives, as similarly outlined above: (i) Evaluate lead impedance trends to understand when an electrode failure occurred or risk of future electrode failure, in order to inform reprogramming and/or to assist with explanations to the clinic, (ii) evaluate patient therapy usage to understand the patient's 1010 normal use patterns and if there has been a change which requires further assessment and potential reprogramming or patient education, and (iii) evaluate the device battery levels and charging history to assist with troubleshooting and educating the patient 1010 on potential charging misuse. When performing proactive care outreach, users 1060, 1070, 1080 may reference device diagnostic statistics as an aid in assessing the patient's situation. In addition to providing proactive care, the user 1060, 1070, 1080 may review device data 1021 periodically for the purposes of ongoing check-in on the patient. Finally, when a patient 1010 calls with an inquiry, the user 1060, 1070, 1080 may refer to the device diagnostic trends and statistics as part of the troubleshooting process with the patient 1010.
4) Generate therapeutic monitoring report(s) as described herein, e.g., in reference to FIG. 8.
5) Generate programming session reports as described herein, e.g., in reference to FIG. 9.

The stage determination as described herein may be performed by the users, and/or on the medical side of the system, e.g., at the secure medical device server 1030 (e.g., components thereof) or by one or more elements between secure medical device server 1030 and portal backend 1050.

FIG. 2 shows a schematic representation of a communication infrastructure 2000 for medical device data allocation to various recipients 2060, 2080 and for determining patient stages on a slightly more detailed level than FIG. 1.

An exemplary medical device 2020 of FIG. 2 provides remote programming data 2130a to a patient remote 2090 and/or receives remote programming data 2130a from the patient remote 2090. The patient remote 2090 may be a patient programmer. The data for remote programming 2130b are further also exchanged between the patient remote 2090 and a (remote) secure medical device server 2030, as well as between the secure medical device server 2030 and a clinician programmer 2100 through remote programming data 2130c. The whole remote programming data transfer 2130a, 2130b, 2130c may be bi-directional and may be transmitted via a continuous communication connection involving the medical device 2020, the remote device 2090, the secure medical device server 2030, and the clinician programmer 2100.

In particular, according to an exemplary workflow for a remote programming session, the remote programming session is started by clinician programmer 2100, which transmits a session request via (remote) secure medical device server 2030 to patient remote 2090. The patient remote 2090 connects to the medical device 2020, e.g. via Bluetooth. In case of a successful connection, the patient remote 2090 returns data to medical device server 2030 which is forwarded to and/or read by clinician programmer 2100. Thereafter, clinician programmer 2100 will send programming data to medical device server 2030, where the data is encrypted and transferred to medical device 2020 via patient remote 2090. By the end of the remote programming session, medical device 2020 will send data via patient remote 2090 to medical device server 2030, where the data is decrypted and whereby data is extracted for clinician programmer 2100 to end the session.

The whole remote programming data transfer 2130a, 2130b, 2130c may be bi-directional and may be transmitted via a continuous communication connection involving the medical device 2020, the remote device 2090, the secure medical device server 2030, and the clinician programmer 2100.

The clinician programmer 2100 may also receive input from a (remote) care professional 2080. The clinician programmer 2100 of FIG. 2 therefore may run a programming application 2101 for the user 2080 to receive and transmit data.

Besides remote programming, as explained herein, traditional in-person programming via the patient remote 2090 may be employed as well, wherein the patient may provide programming data/instructions 2120a to the medical device 2020 via the patient remote 2090. The medical device 2020 may then, in turn, also provide these data 2120a back to the patient remote 2090, e.g., on its current programming (state).

Traditional in-person programming may also occur via the clinician programmer 2100, which, in contrast to the remote programming session outlined herein, may provide the (re-)programming instructions/data 2120d directly to the medical device 2020, e.g., when the clinician programmer 2100 and the medical device 2020 are close together, e.g., during a face-to-face visit of the patient at the care professional's 2080 office/clinic, wherein medical device 2020 and clinician programmer 2100 might be close enough together to exchange data, e.g., via Bluetooth Low Energy, BLE). Besides remote programming, the programming application 2101 of the clinician programmer 2100 in FIG. 2 therefore also offers the opportunity to perform in-person programming.

The clinician programmer 2100 may further send program data 2140c to the secure medical device server 2030 and receive program data 2140c′ from the secure medical device server 2030. For instance, program data 2140c′ can be used to generate a printed follow-up report via (non-medical) output application 2102 which is installed on clinician programmer 2100.

The programming data transfer 2140e shows data transmissions between secure medical device server 2030 and remote monitoring portal 2050. Programming data transfers 2140f, 2140g show data transmissions between the remote monitoring portal 2050 to the device 2062 of recipient 2060 (e.g. a clinician team), and from the remote monitoring portal 2050 to the device 2082 of recipient 2080 (e.g. a representative of the medical device manufacturer).

Further, the medical device provides medical data and/or device data, e.g., device diagnostic data 2110a to the patient remote 2090, and via the patient remote 2090 provides device diagnostic data 2110b to the secure medical device server 2030.

In a first communication path, the secure medical device server 2030 may provide the diagnostic data (not shown) to the clinician programmer 2100, which may additionally or alternatively also receive diagnostic data 2110d directly from the medical device, e.g., during traditional in-person programming 2120d, as described herein. The clinician programmer 2100 of FIG. 2 therefore runs a (non-medical) output application 2102 for the user 2080 to receive any information that may be required for patient monitoring and/or device programming. The clinician programmer may in some examples also forward the directly received diagnostic data 2110d to the secure medical device server 2030 (not shown).

In a second communication path, the secure medical device server 2030 may provide the diagnostic data 2110e along with the program data 2140e to a database, such as remote monitoring portal 2050 which may be accessed in a web-based manner. The remote monitoring portal 2050 may be accessible to different recipients and/or providers 2060, 2080 of data. The recipients and/or providers can, e.g., log into the remote monitoring portal 2050 via a username and password. Once authorized, the respective devices 2062, 2082, of the recipients 2060 (e.g. a clinician team), 2080 (e.g. a representative of the medical device manufacturer) may receive data along a so-called patient pipeline (or patient journey) tracking path 2150f, 2150g, respectively. Along that path, diagnostic data 2110f, 2110g, program data 2140f, 2140g, and patient reported outcome data 2160f, 2160g may be provided to the respective devices 2062, 2082, of the recipients 2060, 2080. The patient reported outcome data 2160h are in the exemplary embodiment of FIG. 2 provided via a patient app on the patient's remote device 2090, but they could also be provided by any other component of the system 2000.

In the exemplary system 2000 of FIG. 2, as explained in reference to FIG. 1, various components may decrypt and/or process data in other ways and/or determine a patient stage within a predetermined patient treatment sequence, as described herein. The components that may execute these functions comprise, e.g., the secure medical device server 2030 (e.g., a component thereof), the portal backend 2050, and/or any component establishing a connection between the medical device server 2030 and the portal backend 2050.

FIG. 3a shows a schematic representation of a communication infrastructure 3000a for medical device data allocation to various recipients 3010, 3060, 3070, 3080 and patient stage determination with a focus on intermediate layers 3030, 3180 managing data transfer and processing between a medical device 3020 and the recipients 3010, 3060, 3070, 3080.

The system comprises a secure medical device server 3030 that may comprise storing means, e.g., a secure medical device database 3032 (such as a hard-drive, a magnetic tape recorder or any other data storage device). It may further comprise computing means 3033 (such as a general purpose or special purpose computer, processor or any other computing device), which may, e.g., receive data from the secure medical device database 3032 and process programming session data and/or medical device data to deliver it in a read-only fashion to any recipient. This may occur e.g., by first feeding the processed data back to the secure medical device database 3032, as shown in FIG. 3a. The medical device database 3032 may, e.g., also store educational material as described herein. From there, (at least partly) processed data and/or educational material may be provided to a connectivity system 3170, e.g., a microservice (server) which transmits the data (e.g., therapy usage, battery history, MRI mode status, and/or lead impedance trend). The processing may for example ensure that the data are present in a form in which they are free of privacy-sensitive information, such that they may leave the medical system 3230/the secure medical device server 3030. The processing may also include a decrypting of the data, as described herein. The secure medical device server 3030 may comprise an interface 3210 for user and account management.

The connectivity system 3170 may, e.g., obtain data via a one-way download from the secure medical device server 3030. It does in general not interface directly with the therapy device such as medical device 3020. Public networks may for example be used for the connection between connectivity system 3170 and secure medical device server 3030 and/or further elements of system 3000a described hereinafter. The connectivity system 3170 microservice, e.g., enables capturing of selected data from the secure medical device server 3030 through one-way data download from the secure medical device database 3032, and formatting of this data into a JavaScript Object Notation (JSON) file, an example for the decrypted medical data comprising a file structure with key-value pairs, wherein the decrypting may also or alternatively ensure that the data are free of privacy-sensitive information, from a regulatory standpoint. The connectivity system 3170 of this exemplary system 3000a does not interface with end users and does not have its own graphical user interface.

The secure medical device database 3032 may also serve as a database for providing data to the gateway 3034 through which data may leave the medical environment 3230 to be transmitted to a data replicator microservice 3035, through which follow-up reports, e.g., on a programming session and/or a data interrogation session may be provided to a business intelligence (BI) layer 3180. As background information, the medical system 3230 may collect medical data comprising therapy and device status information as well as technical monitoring data and transmits this data, e.g. as a package, for example in the form of a Home Monitoring (HM) messages, to the gateway 3034 at a preset time from the medical device 3020. Separately, a search engine in the secure medical device server 3030 may be used for the centralized management of logging information and metrics analysis of multiple patient (medical) devices. The secure medical device database 3032 of FIG. 3a may comprise a NoSQL database. The search engine stores troubleshooting logs and analytics logs from the patient remote 3090, clinician programmer 3100 and secure medical device server 3030. Those may contain information on app usage, such as when certain functions on the patient remote 3090 are used, when programming sessions start, and when the system detects an error (e.g., impedance error that prevents stimulation), etc. The secure medical device server 3030 may be configured to automatically provide a follow-up report to a database/portal backend 3050 upon a programming change of the one or more medical devices. The gateway 3034 may, e.g., comprise a scalable server that securely manages the access of medical data and synchronization of data with any of the devices outside of the medical system 3230 described herein. The data replicator service 3035 may, e.g., comprise means to forward processed medical data it receives from the gateway 3034 to the business intelligence layer 3180, similar to a potential functionality of the connectivity system 3170. Both, the connectivity system 3170 and the data replicator service 3035 may, in the example of FIG. 3a, be outside of the medical system 3230. Outside of the medical system 3230 medical data may be present as described herein. For instance, the medical data is free of privacy-sensitive information, or at least in part free of privacy-sensitive information (e.g. the data may involve Personally identifiable information, PII, and protected health information, PHI).

The device data for a specific patient (with a unique ID) are downloaded daily from both secure medical device server 3030 (e.g. Couchbase and ELK). The input of connectivity system 3170 from Couchbase and ELK may in some examples be simple text in JSON format. The transmitted JSON file contains in this example the last 7 days of data for a patient, or since the most updated association between medical device 3020 and patient remote 3090.

In an example, the data may be provided to connectivity system 3170 in an (at least partially) encrypted manner. The connectivity system 3170 may for example comprise means for receiving (at least partly) encrypted medical data from the secure medical device server 3030 and means for decrypting it. Alternatively or additionally, data from the secure medical device database may be provided to a gateway 3034, where such processing (e.g., decrypting) as described herein may be performed before forwarding the as-processed data via a data replicator service 3035 to the business intelligence layer 3180.

There may be data processing prior to such decryption: The computing means 3033 may process data as described in reference to FIG. 2 and deliver it in a read-only fashion to the business intelligence layer 3180 via the connectivity system 3170 and/or the data replicator service 3035 (which is shown as part of the secure medical device server 3030 but already outside of the medical system 3230 of FIG. 3a). Data handling within the medical system 3230 may be required to fulfill requirements for medical data handling, e.g., in terms of privacy regulations and/or from a regulatory standpoint, while the other components (e.g., the connectivity system 3170, the business intelligence layer 3180, the portal backend 3050, and the user interfaces 3061, 3062) shown in FIG. 3a are considered to be non-medical from a regulatory standpoint. This distinction is transparent from the user end, however, as all components of the system act seamlessly and automatically together. Further, the entire system 3000a becomes available to the users 3010, 3060, 3070, 3080 as soon as they complete a qualification process.

TABLE 2

| Description | Data source |
|---|---|
| Device ID and serial | NoSQL data base |
| number | |
| Lead impedance | NoSQL data base |
| measurement (LIM) | |
| sweep test results* | |
| List of actively used | NoSQL data base |
| electrodes | |
| Battery history log | NoSQL data base |
| Duration of stimulation | Search engine |
| off or each active | |
| therapy program | |
| Counts of stimulation | Search engine |
| on/off changes, program | |
| changes, and amplitude | |
| changes | |
| Statistics of battery and | NoSQL data base |
| stimulation | and Search engine |
| MRI Entry/Exit Log | NoSQL data base |

For each patient, the device data formatted by connectivity system 3170 shown in Table 2 may be stored in a suitable file format. Each of the data fields are described in more detail herein. The secure medical device server 3030 may further be configured to automatically provide a follow-up report to the business intelligence layer 3180 on a programming session, as listed in Table 2, of the one or more medical devices 3020. The follow-up report is 10 forwarded from the business intelligence layer 3180 to database 3050.

The business intelligence layer 3180 may receive these follow-up reports from the data replicator microservice 3035 or it may receive data, e.g., JSON-formatted, or other human- and machine-readable data, from the connectivity system 3170. The connectivity system 3170 may further process the incoming data to provide at least a part thereof in its original or in a modified version. The business intelligence layer 3180 consolidates and stores data delivered by the secure medical device server 3030/the computing means 3033, the data replicator microservice 3035 and the connectivity system 3170 in a cloud data warehouse/database 3182. The business intelligence layer 3180 performs minor data processing, e.g., by the means for data processing 3181, wherein the means for data processing 3181 may be configured to process the data from the connectivity system 3170 and/or from data replicator microservice 3035 at least partly before and/or after decryption/decoding, preferably for diagnostic identification of issues, for providing visualization data for visualization of the data and/or to make the data clinically understandable.

The system 3000a may further comprise means for triggering an alert, based at least in part on the medical data, and/or the subjective and/or objective data, as described herein, based on medical issue/diagnostic data output and/or non-medical simple data transformations, e.g., controlled and/or managed by the (non-medical) portal 3050. The business intelligence layer 3180 may also comprise means for data processing 3181 to make data from the connectivity system 3170 and/or from data replicator microservice 3035 readable (in addition or alternatively to respective means in connectivity system 3170 and/or in data replicator microservice 3035). It may further comprise a cloud data warehouse/database 3182 to store data before and/or after processing and/or in between processing steps, and separate computing means for providing visualization data 3183 for visualization of the data.

The business intelligence layer 3180 is, in the example of FIG. 3a, further connected to a technical monitoring device 3220 providing (selected) data to said technical monitoring device 3220 for technical monitoring of the whole system 3000a and/or any of the parts thereof described herein.

Independent of the exact distribution of computing and storing means in the business intelligence layer 3180, the following functions may be performed therein: The business intelligence layer 3180 performs basic arithmetic calculations such as basic logic, addition, subtraction and averaging, time aggregation and filtering of data (e.g. therapy usage within a patient's day) to the data it received. After the business intelligence layer 3180 processes it, the data is routed to and displayed in the health cloud/the portal backend 3050 for viewing by users 3010, 3060, 3070, 3080. The business intelligence layer 3180 may comprise a cloud data warehouse 3182 and a (cloud) analytics tool 3183. The data warehouse 3182 may, e.g., also store educational material as described herein. Patient reported outcomes (surveys etc.) entered on the patient app may be transmitted to the business intelligence layer 3180 through the portal backend 3050. Medical data from secure medical device server 3030/the computing means 3033, the data replicator microservice 3035 and the connectivity system 3170 may be transmitted directly to the cloud data warehouse 3182.

The portal backend 3050 may, e.g., provide a first data request requesting data from the system 3000a. Any other component of said system 3000a may comprise means for receiving the first data request from the database/portal backend 3050. Further, the system 3000a may comprise means for sending a second data request to the secure medical device server, preferably based at least in part on the first data request. For example, the means for receiving and/or sending the first and second data request may be comprised in the business intelligence layer 3180 and/or the secure medical device server 3030.

The system 3000a may further comprise means for providing medical data at least partly based on the second data request. The means for providing may be located at only one position or at any position in the system 3000a, where data may be requested from to provide the medical data on-demand and/or in response to the second and/or the first data request, e.g., in the medical device 3020, the secure medical device server 3030, and/or the business intelligence layer 3180.

Two examples of the business intelligence layer 3180 calculations include, referring in these examples to a SCS device as a medical device:
1) Therapy Usage Percentage: The connectivity system 3170 interface provides a therapy usage percentage per program, per day. As new daily data is being loaded into the cloud data warehouse 3182, the system 3000a will calculate and update a rolling 7-day average and a 30-day average of Therapy Usage and store these values in two designated fields in the data warehouse 3182. Alternatively, 90-day average and 180-day average of Therapy Usage can be stored.
2) Active Electrode “Out of Range”: The connectivity system 3170 provides an array of the measured lead impedances for all electrodes and a list of electrodes used in each therapy program. The business intelligence layer 3180 iterates through the array of measured lead impedances and
   identifies if any are used in the active or most recently valid and active program and
   identifies those lead impedances flagged as out of range.
The resulting list of electrodes with impedance out of range is then stored as a field in the data warehouse 3182 and represents the active electrodes which are out of range. The fields will then be displayed in the portal backend 3050 screens.

Monitoring users 3060, 3070, 3080 may access all data directly in the business intelligence layer 3180 via login to the cloud analytics tool 3183 (e.g., Qlik Sense®). The cloud analytics tool 3183 may, e.g., be an off-the-shelf analytics software used for self-service visualization, interactive dashboards, reporting as well as advanced search capability. For example, cloud analytics tool 3183 access may not be used for day-to-day remote monitoring operations by the care team 3070, 3080 nor by health care professionals 3060.

The portal backend 3050 may comprise business logic 3051 which may include a database 3052 of patients and their proactive care triggers as determined by the data warehouse 3182 and, e.g., optional tools like a cloud voice solution embedded in the portal backend (not shown). The database 3052 may, e.g., also store educational material as described herein. The purpose of the portal backend 3180 is to provide an access point for any authorized recipients 3010, 3060, 3070, 3080, as described herein. They may access via a user interface displayed on their devices 3061, 3081, 3090, e.g., also directly through a patient app run by the patient remote 3090, e.g., a mobile phone, or via a health cloud portal and/or a provider portal.

The portal backend 3050 may, e.g., as shown in FIG. 3a, be protected from unauthorized accessing via a login capability 3200 (e.g., as implemented by standard in the portal backend 3050). Data may be provided from the business intelligence layer 3180 to the portal backend/database 3050 as decrypted and/or processed medical data, patient management data, and/or device-specific data which may be available along with each other in a useful way for any authorized user to be received and understood.

For example, the business intelligence layer 3180 may comprise means for receiving encrypted medical data from a secure medical device server 3030, means for decrypting the medical data at least partly, and means for providing the at least partly decrypted data to a database, wherein the database may be the database of the portal backend 3050. The encrypted medical data may, e.g., comprise measurement and/or operational data of an at least partly implantable medical device. Additionally or alternatively, the connectivity system 3170 may comprise the means for receiving, means for decrypting, and means for providing. Further, also the computing means 3033 may comprise the means for receiving, means for decrypting, and means for providing. The computing means 3033 is comprised in the secure medical device server 3030 and receives data from the secure medical device database 3032 of the medical device server 3030. The means for receiving, means for decrypting, and means for providing may also be distributed in said system 3000a in any other way.

FIG. 3b shows a schematic representation of a different communication infrastructure 3000b for medical device data allocation to various recipients 3020, 3060, 3070, 3080 with a focus on the intermediate layers managing data transfer and processing between the medial device 3020 and the recipients 3020, 3060, 3070, 3080.

The at least one medical device 3020, the patient remote 3090, and the clinician programmer 3100 are configured and connected to the system 3000b as described herein.

The secure medical device server 3030 may comprise differences compared to the one described with reference to the exemplary embodiment shown in FIG. 3a.

The secure medical device server 3030 of FIG. 3b may comprise a database 3032b, such as a NoSQL database, operably connected to a data forwarding microservice 3037 which is configured to, e.g., forward patient (remote) notifications, clinician (programmer) notifications, and/or follow-up reports (as described herein) towards the portal backend 3050. It may comprise, e.g., computing means 3033 and/or the connectivity system 3170, both, e.g., as described herein.

The data forwarding microservice 3037 may provide either of the following data transfer paths: clinically understandable interface database 3038 and/or gateway 3034, as described herein. These two transfer paths may be alternatives to one another or be comprised both in the secure medical device server 3030 of FIG. 3b. They may provide data, e.g., in the form of follow-up reports to the business intelligence layer 3180.

The business intelligence layer 3180 of FIG. 3b may be adapted similarly to that of FIG. 3a

The portal backend 3050 of FIG. 3b may comprise differences compared to the one described with reference to FIG. 3a, especially in form of additional functionalities and subunits: It comprises a logic and presentation layer 3055 and a means for user and account management 3056, wherein these two components 3055, 3056 exchange, e.g., user/account data and/or medical data. For example, the patient may, via the patient remote 3090, provide further data, for example pain scores, to the database of the logic and presentation layer 3055 and receive data like for example pain maps, journey information, and/or educational materials from the database of the logic and presentation layer 3055. The portal backend 3050 may transmit data, e.g., clinical information from the logic and presentation layer 3055 to the users 3060, 3070, 3080 and/or identification/user account information from the users 3060, 3070, 3080 to the logic and presentation layer 3055. All users 3010, 3060, 3070, 3080 may log in to the portal backend 3050 via a login capability 3200 (e.g., as implemented by standard in a portal backend 3050), as described herein.

There may, e.g., as shown in FIG. 3b, be an exchange of synchronization information 3057 between the portal backend 3050 and the secure medical device server 3030. First synchronization information available in the portal backend 3050 may, e.g., be transmitted to the means for user and account management 3056 of the portal backend and, via the exchange of synchronization information 3057, to the secure medical device server 3030, wherein they may be available as second synchronization information which may be provided, e.g., to a database 3032b of the secure medical device server 3030 and comprise at least some of the first synchronization information such as to ensure that at least some of the same information required for synchronization of the portal backend 3050 and the secure medical device server 3030 are available at both ends of the system 3000b. The synchronization information may, e.g., comprise information regarding user account relationships like login data, usernames, passwords, etc. Which may be critical for user authorization. Therefore, a synchronous operation of the portal backend 3050, where a user may request authorization, and the secure medical device server 3030, from where they may request medical data, is required for a safe and reliable operation of the whole system 3000b.

In comparison, FIG. 3a and FIG. 3b illustrate that any of the components and/or functionalities described herein may be transferred fully or in part to another part of the system 3000a, 3000b. The same applies to the systems described in reference to the other Figures as well.

In the exemplary systems 3000a, 3000b of FIGS. 3a and 3b, and as explained in reference to FIGS. 1 and 2, various components may decrypt and/or process data in other ways and/or determine a patient stage within a predetermined patient treatment sequence, as described herein. The components that may execute these functions comprise, e.g., the computing means 3032, the portal backend 3050, and/or any connectivity system 3170. Either of these components may, e.g., receive the medical data required for performing the respective functionality and optionally also the criteria required to define/determine the patient stage depending on whether the medical data of the patient match said criteria. The medical data and/or criteria of the stages may, e.g., be stored in any of the databases described herein and be made available to the component determining the stage when required. The second database described herein may, e.g., comprise the cloud data warehouse/database 3182 and/or the database 3052 of the portal backend 3050.

FIG. 4 shows a schematic representation of a part of the communication infrastructure 4000 for medical device data allocation to various recipients as well as stage determination with a focus on the architecture and internal data transfer of the intermediate layers 4050, 4180, 4200 managing data transfer and data processing:

On a high level, the system 4000 comprises three blocks: (i) the extended medical system 4200, (ii) the business intelligence layer 4180, and (iii) the portal backend 4050. The extended medical system 4200 may comprise the medical system 3230 as described with reference to FIG. 3a.

The extended medical system 4200 may comprise a connectivity system 4170 that may be similar to connectivity system 3170 of FIG. 3a. The extended medical system 4200 of FIG. 4 essentially corresponds to the medical system of FIGS. 3a and 3b plus the connectivity system 4170, as implied by the term “extended”. This grouping, however, may be seen as a mere label. Also, it may comprise a data replicator service 4035 which may be similar to that of FIG. 3a. It serves the purpose of providing non-medical (from a regulatory standpoint) data to the business intelligence layer 4180 which may be similar to business intelligence layer 3180.

The business intelligence layer 4180 of FIG. 4, e.g., receives and stores data for relevant patients, performs logic/calculations to provide only the requested data to portal 4050. Further, it may unify the formats of data received from the connectivity system 4170 and the data replicator service 4035 to appropriate types used by calculations needed by portal backend 4050.

The portal backend 4050 allows remote care team to create reports and/or queries for patient(s) and to define follow-up tasks for patients based on reports, stores all patients and their proactive care triggers, wherein all new data are received from a cloud data warehouse/database 4182 of the business intelligence layer 4180, and generates a unique patient ID at the beginning of a patient journey to support identifying the cloud data warehouse/database 4182 data originating from the secure medical device server 4030.

Exemplary embodiments of the above blocks 4050, 4180, 4200 are described in the following in more detail:

The exemplary secure medical device database of FIG. 4 comprises a search engine 4032a (comprising a database) for management of logging information and metrics analysis of multiple devices, an on-site and/or cloud-based, distributed database 4032b, (e.g., provided NoSQL), and a computing means 4033. One of the functionalities of the computing means 4033 is to process and/or transform follow-up data and exchange them with the database 4032b. Both, the search engine stack 4032a and/or the database 4032b may provide data to the connectivity system 4170 and/or provide data to the data replicator service 4035, e.g., requiring a step of providing authorization for receiving said data, for example by requesting a log-in via a username and a password or another key. This may analogously apply to any other connection and/or data transfer described herein.

The connectivity system 4170 and/or the data replicator service 4035 of FIG. 4 are configured to then transmit the data at least partly in their original or in a modified form to the business intelligence layer 4180, e.g., via a secure VPN connection and/or as described herein.

The data sent by replicator service 4035 may, e.g., comprise a follow-up report as described herein. Such exemplary follow-up reporting may, e.g., work according to the workflow described in the following:
1. A follow-up (FUP) is provided (either local or remotely using a clinician programmer)
2. An HM message with that FUP information is sent to the secure medical device server 4030 (where it creates an entity “Follow-up” in the Couchbase database 4032b)
3. The system “listens” for a FUP not having changed for, e.g., at least 15 minutes:
   a. In the embodiment described here includes a flag indicating the FUP is done, so the system 4000 knows that the FUP is done definitively.
   b. Due to offline considerations, the FollowUp object can be updated in the far future.
4. Once a FUP is idle for, e.g., 15 mins or more, the secure medical device server 4030 generates a clinically understandable “FollowupReport”.
5. The FollowupReports gets pushed to the data warehouse 4182 via the means for data processing 4181 acting as a bucket;
6. The FollowupReport consists of:
   a. Patient meta data (name, lead info, etc.)
   b. Settings of stimulator (stimulation: on/off, MRI mode, remote services, etc.)
   c. All therapy changes made in follow-up (initial and final settings)
   d. All clinician programmer user notifications
7. Snowflake contains an application programming interface (API) that parses the FollowupReport and picks out the data relevant for the PDFs to be created.
8. The portal backend 4050 performs batch processing (of FUPs) e.g. on a daily basis (other cadences possible, as for instance twice a day, every hour).

In an exemplary embodiment of this workflow relating to follow up reporting, the data warehouse 4182 may compare the FollowupReports from at least 1 calendar day. For example: When there are 4 FUPs with: day1=FUP1, FUP2; day2=FUP3, and FUP4. The API for generating the reports only provides the initial/final settings for a patient per calendar day, effectively ignoring intra-day details, by reporting only day-to-day changes.

The data from replicator service 4035 and connectivity system 4170 are then received by means for data processing 4181 of the business intelligence layer 4180. The means for data processing 4181 may, e.g., be a virtual private cloud (VPC), a secure, isolated, private cloud hosted in a public cloud. means for data processing 4181 can run code, store data, host websites and perform further functions. VPCs may be advantageous as they combine the scalability and convenience of public cloud computing with the data isolation of private cloud computing. In detail, the means for data processing 4181 may comprise a first unit for data processing 4181a and a second unit for data processing 4181b, wherein the first unit for data processing 4181a receives the data from the connectivity system 4170 and the second unit for data processing 4181b receives the data from the data replicator service 4035. The first and second units for data processing 4181a, 4181b may then process the data as described herein to provide processed data to a database/data warehouse 4182, e.g., a cloud data warehouse, e.g., via a private link. The data warehouse 4182 comprises data ready to be provided to the portal 4050 and/or to a (cloud) analytics tool 4183, e.g., in the exemplary system 4000 of FIG. 4, a tool 4183 for providing visualization data for visualization of the data that may be received from the data warehouse 4182. The visualization tool 4183 may comprise, e.g., external software like Qlik Sense® (a Cloud BI analytics tool) 4183a and visualization database 4183b, e.g., for internal and/or intermediate storing of (at least partly) processed data, unprocessed data received from the data warehouse 4182 and/or predetermined building blocks for visualizations and/or reports, etc.

The portal backend of FIG. 4 comprises (e.g., a Salesforce.com) business logic 4051 and a database 4052, e.g., of all patients, their proactive care triggers, and/or reports generated by the system and/or involved technical staff and/or health professionals. The business logic 4051 and the database 4052 are operably coupled such that the business logic may perform function on and/or based on the data in the database 4052. These functions may also comprise requesting/pulling data from the business intelligence layer 4180, in detail the data warehouse 4182, and/or from deeper databases, e.g., in the extended medical system 4200.

As shown in FIG. 4, the output of the tool 4183 for providing visualization data may be routed to the portal backend 4050 via a corporate IT device 4240. Further, also the clinician programmer 4100 and/or a provider IT device 4260 may be in bidirectional exchange with the portal backend 4050, wherein the clinician programmer 4100 itself is in connection with a respective (e.g., cloud-based) system for synchronization 4250, e.g., of clinician programmer settings. Last, the patient app 4040 may be used for inputting patient-reported data into the system 4000 via the portal backend 4050 and/or for receiving data from the portal backend 4050 (not shown).

The business intelligence layer 4180 may further exchange any data (besides with the visualization tool 4183) via unidirectional or bidirectional communication connections with any finance, logistics, and/or compliance backends for overall workflow management (not shown). Such backends may comprise one core backend operably connected to further units and/or the respective units may communicate directly with the business intelligence layer 4180. The units, irrespective of the exact connection to the business intelligence layer 4180 may comprise one or more complaints analysis networks, medical device reporting interfaces (e.g., to state institutions), field inventory management systems, trade services (e.g., SAP Global Trade Services), sales and use tax management systems, compliance systems (e.g., Medispend), logistics services (e.g., Mobisys), and/or commissions systems (e.g., SAP and/or usable via mobile app(s)).

FIG. 5 shows an abstracted schematic representation of the communication infrastructure 5000 for medical device data allocation to a portal backend 5050 and stage determination as described similar to those in FIGS. 1-4. In this representation, many intermediate and/or internal elements of the system 5000 are not shown to provide a better overview and indeed in some embodiments only one or more selected such elements are present. These elements are described elsewhere herein and may be incorporated in the system 5000 of FIG. 5.

The first block 5300 in the simplified schematic representation of FIG. 5 comprises the medical device 5020 and the secure medical device server 5030, wherein the medical device 5020 provides (encrypted) medical data to the secure medical device server 5030. The medical data may comprise, e.g., a home monitoring frame containing different data, many of which are highly sensitive from a privacy/regulatory perspective. For example, these data comprise patient information (sensitive), a device status, battery and charging information, therapy information, ISW forensic data, and/or physiologic monitoring data. Typically, these data are compressed and encrypted to minimize the required medical device memory. Especially IMDs have only limited storage due to limitations in terms of available space and energy consumption that large data storage may bring along. The secure medical device server 5030 processes, decompresses, and/or decrypts the compressed and/or encrypted medical data at least partly.

The second block 5400 comprises a connectivity system 5170 and an business intelligence layer 5180.

At least a part of the medical data at least partly decrypted by the secure medical device server 5030 may be transmitted to the second block 5400 (in detail, its connectivity system 5170).

Additionally or alternatively, a follow-up report, as described herein, may be provided by the secure medical device server to the second block 5400 (in detail, its business intelligence layer 5180).

The data provided to the connectivity system 5170 may, e.g., have the following content (or other, e.g., non-human-readable content/format):
54 68 65 20 71 75 69 63
6b 20 62 72 6f 77 6e 20
66 6f 78 20 6a 75 6d 70
73 20 6f 76 65 72 20 31
33 20 6c 61 7a 79 20 64
6f 67 73 2e

In general, a proprietary and/or compressed format may be used.

The business intelligence layer 5180 may additionally receive data from the connectivity system 5170, and (optionally) further data from a patient remote 5090 and/or further devices 5270, e.g., wearables, web-user-interfaces, etc. The further data may comprise patient-reported data, health professional-reported data, and/or device-reported data, e.g., recorded by the at least one external device 5090, 5270. The further data from the further devices 5090, 5270 may comprise, e.g., automatically acquired data from external devices, physiological monitoring data, e.g., from wearables like activity monitors, step counters, pulse frequency, etc., patient-reported data from, e.g., surveys via the patient app, and/or clinician-reported data. The further data may generally comprise objective and/or subjective data.

The business intelligence layer 5180 processes and merges all incoming data at least partly and provides processed data to portal backend 5050, e.g., as required to be outputted by the portal backend 5050.

The processed data preferably have a human- and/or machine-readable format and content, e.g., in form of a JSON file. The processed data 5184 may, e.g., have the following content (or other, e.g., human- and/or machine-readable content/format):

```
{
  therapyDate=2023-03-01
  patientID=“P-0123”
  therapyUsage=“100%”
  BatteryStateOfCharge=75
  painScore=3
}
```

In general, thus the data may be decrypted from a proprietary format to a generally readable (data) format, such as JSON or similar formats, for example.

The portal backend 5050 of the example shown in FIG. 5 does not perform any further data transformation but just configures the visualization of the received pre-processed data 5184. For example, the portal backend 5050 may output sales data, patient data, stimulator data, follow up data, patient management data (including, e.g., educational information for education sessions and/or proactive care triggers), and/or further clinically understandable data. Generally, all data received and/or outputted by the portal backend 5050 via a user interface maybe clinically understandable to any expert on the respective medical field and/or on the respective medical device(s) 5020, 5270. The portal backend 5050 performs functions to visualize the clinically understandable data in a one stop shop for different recipients. For example, this may facilitate sales, patient journey management, and remote monitoring with proactive care by health professionals.

In some examples, the business intelligence layer 5180 may decrypt encrypted data received from the secure medical device server and/or a secure medical device database located therein.

In some examples, the business intelligence layer may determine one or more patient stages at least in part based on the incoming data as outlined herein.

FIG. 6a shows an exemplary graphical user-interface 6000a related to the portal backend of the system as described with reference to FIGS. 1-5. The user-interface 6000a displays information in reference to a patient journey 6100, wherein past/completed stages 6110, the current state 6120, and/or upcoming/future stages 6130 are shown for a complete overview of the patient journey 6100. Notably, the patient journey may comprise a phase before the patient has an implant or a trial device, a trial phase in which the patient has a trial device and a monitoring phase in which the patient has received their device, e.g., an implanted medical device providing data that is output at least in part by the user interface 6000a. The exemplary user-interface 6000a of FIG. 6a shows (on the bottom left) an event log of events that occurred during the trial phase 6300 and during the pre-trial phase 6200, and it may in other examples also show events that occurred in other phases. On the right, the user-interface 6000a shows notifications 6400 on upcoming and overdue events as well as on past events. These notifications 6400 may be provided to the patient and/or any involved device or health expert for workflow management.

FIG. 6b shows another version of the exemplary graphical user-interface 6000b related to the portal backend of the system. Like in FIG. 6a, the user-interface 6000b displays information in reference to a patient journey 6100, wherein past/completed stages 6110, the current state 6120, and/or upcoming/future stages 6130 are shown for a complete overview of the patient journey 6100. The patient journey 6100 may comprise a phase before the patient has an implant or a trial device, a trial phase in which the patient has a trial device and a monitoring phase in which the patient has received their device, e.g., an implanted medical device providing data that may be output at least in part by the user interface 6000b. In contrast to FIG. 6a, the exemplary user-interface 6000b of FIG. 6b shows no log of events that occurred during the trial phase 6300 and during the pre-trial phase 6200, but patient information, such as general demographics 6500, e.g., comprising an account name, a preferred name, a patient identification, a patient status, a reason for inactivity, a status reason code, a link to/information on a therapeutic report schedule, a primary/secondary language of the patient and/or the clinician(s) involved, an account owner, a journey stage, patient contact information, patient gender, patient age, patient birth date, and/or an account source, etc.

On the right, the user-interface 6000b shows notifications 6400 on upcoming and overdue events as well as on past events. These notifications 6400 may be provided to the patient and/or any involved device or health expert for workflow management, similarly, as outlined with reference to FIG. 6a.

FIG. 6a and FIG. 6b may be two different pages/views of the same user interface, e.g., of the patient app run by a mobile phone of the patient or any other suitable patient device.

Other views may, instead of a log of events or general demographics (as in FIGS. 6a and 6b), show information on pain history, a care team, clinical reports, trends, and statistics, etc.

Wherever data are transmitted in described systems and/or methods, the data may be present as raw data and/or data reports may be transmitted. As described herein, e.g., with reference to FIG. 1, the system may provide reports, e.g., on a trial phase, wherein the patient tested a trial medical device (see FIG. 7), on therapeutic monitoring (see FIG. 8) and/or on programming sessions, wherein, the medical device was (re-)programmed (see FIG. 9). The following relates to exemplary reports relating to scenarios where the relevant medical device is an SCS.

FIG. 7 shows an exemplary trial summary report 7000. Such report 7000 may be issued by the system automatically or upon request by, e.g., the attending clinician. At the end of an SCS trial period (i.e., to determine if a patient is a suitable candidate for an implanted SCS device), patient reported metrics may be summarized in a trial summary report 7000 which may be reviewed by a clinician user as supplementary information to assess the patient's response to the SCS therapy.

The following is an exemplary workflow to generate the trial summary report 7000: (i) The trial summary report 7000 is generated in the portal by the care team after the patient has completed their SCS trial. (ii) During the lead pull appointment, the attending clinician/technical expert/manufacturer representative may add pain relief and notes (including qualitative description of medication changes) to the report 7000 based on the patient's final assessment of their therapy response. (iii) The manufacturer representatives may make the report available to the clinic in the portal. (iv) The clinician may use the portal to access the report 7000.

The exemplary report 7000 of FIG. 7 comprises a report identification section 7100, e.g., a title relating to the topic of the report 7000, a patient identification section 7200, e.g., comprising patient name, date of birth, manufacturer identification, implantation date, medical device (e.g., SCS) serial number, and/or medical device (e.g., SCS) model. The exemplary trial summary report 7000 comprises, in this specific example, six further sections 7300, 7400, 7500, 7600, 7700, 7800. Section 7300 comprises a pain score trend in which the pain score is shown as a function of time, for example as data points on multiple consecutive days to visualize trends during the trial phase. Section 7400 comprises a body map highlighting primary pain areas. As shown in FIG. 7, primary areas where the patient experienced pain may be highlighted in a color in a front and back view of a body map of the patient. Further sections 7500, 7600, 7700, 7800 show medical data as a function of time for example as single data points on different dates, namely function scores (section 7500), sleep scores (section 7600), patient activity (section 7700), and patient mood scores (section 7800).

FIG. 8 shows an exemplary therapeutic monitoring report 8000. Such report 8000 may be issued by the system automatically or upon request by, e.g., the attending clinician. Issuing such therapeutic monitoring reports 8000, similar, or other reports for remote monitoring of patients may be seen as a central functionality of the system itself as it allows for closed loop monitoring of the patient which may significantly improve remote care quality, especially considering the triggers implemented in the system as described herein. The portal may allow clinician users to access a therapeutic monitoring report 8000 on-demand to review the patient's condition with the patient during in-office or remote visit. The therapeutic monitoring report 8000 may contain device data information captured in the portal and can be downloaded by the clinician for documentation purposes. Furthermore, the therapeutic monitoring report 8000 may serve as evidence of remote patient monitoring when submitting reimbursement claims for remote therapeutic monitoring reimbursement codes. Clinician users may be able to adjust the report data time window to capture a minimum of 16 days of device data within a 30-day period to meet criteria for reimbursement for remote therapeutic monitoring and for physiologic monitoring, respectively. The portal may also be capable of logging the number of report generations per month for internal tracking. Further, the system may be capable of alerting the clinic if the minimum of 16 days of device data within a 30-day period is at risk of not being met.

The exemplary report 8000 of FIG. 8 comprises a report identification section 8100, e.g., a title relating to the topic of the report 8000, a patient identification section 8200, e.g., comprising patient name, date of birth, manufacturer identification, implantation date, medical device (e.g., SCS) serial number, and/or medical device (e.g., SCS) model. The exemplary therapeutic monitoring report 8000 comprises a first section 8300 showing daily therapy usage detail trends in a given time window, where in the therapy usage of the patient by using the medical device is shown overtime for example by showing which program was used or not used on each day. Each color of the respective bar for one time window, e.g., a day, indicates a program/stimulation mode. The therapeutic monitoring report 8000 further comprises a section 8400 comprising a patient interaction log listing which triggers were activated on which date which technical staff member was involved and how the associated issue was resolved. Section 8500 of the (remote) therapeutic monitoring report 8000 comprises information on the programs in the patient programmer on a given date, for example the date of issuing the therapeutic monitoring report 8000. Section 8500 may, e.g., further comprise for each program identified by a program name, the contacts involved in delivering the therapy and the respective stimulation mode. Section 8600 of the exemplary report 8000 comprises information on therapy usage mix, for example in a 30-day time window. In the example of FIG. 8, two pie charts show the relative therapy usage by program name and by stimulation mode.

FIG. 9 shows an exemplary programming session report 9000. Such report 9000 may be issued by the system automatically or upon request by, e.g., the attending clinician. For each therapy device programming session interaction, the care team and clinicians may view the programming session report 9000 in the portal.

The exemplary report 9000 of FIG. 9 comprises a report identification section 9100, e.g., a title relating to the topic of the report 9000, a patient identification section 9200, e.g., comprising patient name, date of birth, manufacturer identification, implantation date, medical device (e.g., SCS) serial number, and/or medical device (e.g., SCS) model. The report 9000 of FIG. 9 further comprises a programming section 9300, e.g., comprising a follow-up date, a programming duration, information on who performed the programming, reason for the follow up, and/or a location of the programming session. Such programming section 9300 may relate to a (re-)programming of the medical device of the patient either via conventional in person programming or via remote programming. Further, it may comprise a general settings section 9300, listing, e.g., information on the stimulation mode, MRI mode, and/or lead impedance tests. The system may issue a programming session report 9000 independently of how exactly the programming has been performed. The exemplary programming session report 9000 further comprises two sections 9500, 9600 on the two leads (A and B) of the SCS, e.g., relating for each contact to an impedance and an electrode status. The report 9000 further comprises a program summary comprising a first chart 9700 on the initial settings of the SCS and a second chart 9800 on the final settings of the SCS. In the shown example, for each program of the SCS, indicated by a program name, the respective chart 9700, 9800 indicates whether the program has been edited (changed/newly created/deleted/ . . . ), its mode, which contacts are involved in the respective stimulation, the stimulation strength/amplitude (e.g., in mA), the pulse width (e.g., in microseconds) and/or the stimulation frequency (e.g., in Hz).

FIG. 10 shows a flow diagram of the preferred embodiment of a patient triage and automatic journey sequencer of the patient journey 10000. In this flow chart, every step marked by a star comprises a success-check such that only upon completion of the respective step, the patient may progress in their journey 10000 to the next step. Every step marked by a triangle comprises a success check such that the progress is paused if that step is not completed successfully until the underlying issue is rectified. Steps marked by an octagon indicate pause steps at which patients may leave the patient journey 10000/be stopped in their patient journey 10000 when that respective step fails (e.g., data cannot be validated/are incorrect).

Thereby, these logic checks provide a basis for (automatic) guidance of the patient along the patient journey 10000 which may be implemented in the system as described herein.

The patient journey 10000 starts with step 10100, wherein the patient enters the data stream through download of a patient application which may be run, e.g., by the patient remote/the patient's mobile phone or any other suitable device, and registration, or by registration into the portal, as described herein, by an assistant, e.g., a clinician.

This start 10100 initiates the pre-trial phase 10200 which comprises, for example, the four steps shown in FIG. 10:
a) step 10210 For providing patient education and collecting baseline information, like for example fundamental patient data like gender, weight, age, etc.,
b) step 10220 for validating said information (or not),
c) step 10230 for scheduling and performing a psychological evaluation of the patient by a qualified health professional who may for example enter the results into the system via the portal as described herein, and
d) step 10240 for obtaining authorization prior to the trial period.

Upon obtaining authorization prior to the trial period 10240, the medical intervention trial period or trial phase 10300 Is initiated in which the patient may for example test a trial medical device for a limited time. When this phase 10300 is completed successfully, e.g., sufficient data is collected and provided to the attending clinician, follow up and outcome report may be issued 10310. Should follow up and/or outcome report indicate that the patient is not suited to receive a permanent implant, the progress may be stopped here. Otherwise, the patient is moved to step 10320 in which he or she may receive prior authorization before receiving a permanent implant in step 10330, e.g. an SCS device in a respective operation. A post operation follow-up report 10340 may be issued via the system. When that report 10340 indicates a successful implantation, the patient may progress (automatically) to the long-term follow-up phase 10400 comprising a closed loop of the following three steps:
a) step 10410 of continuously and/or automatically monitoring data acquired by the medical device of the patient,
b) step 10420 of monitoring triggers and/or a regular (e.g., monthly) cycle, and
c) step 10430 of supporting follow-ups and/or reports on steps 10410 and 10420, as described herein.

Steps 10410, 10420, 10430 may be repeated one after another in a closed loop 10400 for as long as required for continuous remote monitoring of the patient.

Any of the data described herein in reference to the patient journey 10000 may be collected, provided, decrypted, visualized, and/or processed otherwise by the system described herein. Progression of the patient along their journey 10000 as described for example in FIG. 10 may occur automatically and/or controlled by said system and/or at least partly controlled by one or more attending clinicians and or the patient themselves, e.g., via the patient app and/or the portal backend of the system.

FIG. 11a shows an exemplary device data transmission log 11000a. This data log may be provided, e.g., to the portal backend as described herein to be accessed by a clinician to track the data received through the system and/or to assign the data to potentially multiple patients and/or devices. The log 11000a may comprise a list of data transmission events, e.g., providing for each event the serial number of the involved medical device, a message creation date, a message creation time including the relevant time zone, and/or the time at which the data were received by the portal backend.

FIG. 11b shows a user interface 11000b for data input for the identification of primary pain areas 11110. The patient and/or the clinician may provide corresponding data on the type of pain 11300 (e.g., aching, burning, cramping, shooting, tingling, numbness, stabbing, spasm, etc.) and/or a pain intensity 11200 (e.g., on a scale from 0-10) for different areas 11110 of the patient's body according to a body map 11100. This interface 11000b may be e.g., shown like this or in a similar way by the patient app and/or the (online) portal backend described herein.

FIG. 11c shows a long-term follow-up display 11000c of a medical device battery charge 11400 and a recent recharge 114100, which may be e.g., shown like this or in a similar way by the patient app and/or the (online) portal backend described herein. The exemplary graph shows three data series, the medical device battery charge 11400, the default charge level of 15% 11500, and the low battery level of 10% (both of which may be triggers for alters provided to e.g., the patient via the portal backend and/or the patient app instructing the patient to recharge the medical device) over time. Upon the recent recharge 114100, as possibly suggested by the respective alert triggered by the battery charge falling below 15% and/or 10%, the battery charge increases from <5% to 100%.

FIG. 12a shows a flow diagram 21000a linking the current patient state in the patient journey with educational material provided to the patient according to the patient state. The patient state may, in one simple exemplary embodiment, be defined by the phase of the patient journey, e.g., the introduction (pre-trial) phase 12200, the trial phase 12300, and the active therapy phase 12400. The patient app may then be configured to provide educational material 12500a, 12500b, 12500c matching the respective phase 12200, 12300, 12400. In the example of FIG. 12a, the educational material 12500a may be provided when the patient is in phase 12200, the educational material 12500b may be provided when the patient is in phase 12300, and the educational material 12500c may be provided when the patient is in phase 12400. Providing the educational material 12500a, 12500b, 12500c may further be adapted to sub-steps of the respective phase 12200, 12300, 12400, as described in reference to FIG. 10.

FIG. 12b shows a graphical user-interface 12000b of an exemplary patient app 12040 run by the patient remote 12090 showing selected educational material based at least in part on the patient state in the patient journey (see FIG. 10 and FIG. 12a). In the current state of the patient, the patient app 12040 provides the exemplary educational material 12500 to the patient to be selected/viewed by the patient.

The methods according to the present invention may be implemented in terms of a computer program which may be executed on any suitable data processing device comprising means (e.g., a memory and one or more processors operatively coupled to the memory) being configured accordingly. The computer program may be stored as computer-executable instructions on a non-transitory computer-readable medium. A data processing device may comprise one or more general purpose and/or specific purpose processors, etc.

Embodiments of the present disclosure may be realized in any of various forms. For example, in some embodiments, the present invention may be realized as a computer-implemented method, a computer-readable memory medium, or a computer system.

In some embodiments, a non-transitory computer-readable memory medium may be configured so that it stores program instructions and/or data, where the program instructions, if executed by a computer system, cause the computer system to perform a method, e.g., any of the method embodiments described herein, or, any combination of the method embodiments described herein, or, any subset of any of the method embodiments described herein, or, any combination of such subsets.

In some embodiments, a computing device may be configured to include a processor (or a set of processors) and a memory medium, where the memory medium stores program instructions, where the processor is configured to read and execute the program instructions from the memory medium, where the program instructions are executable to implement any of the various method embodiments described herein (or, any combination of the method embodiments described herein, or, any subset of any of the method embodiments described herein, or, any combination of such subsets). The device may be realized in any of various forms.

Although specific embodiments have been described above, these embodiments are not intended to limit the scope of the present disclosure, even where only a single embodiment is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed herein (either explicitly or implicitly), or any generalization thereof, whether or not it mitigates any or all of the problems addressed herein. In particular, with reference to the appended claims, features from dependent claims may be combined with those of the independent claims and features from respective independent claims may be combined in any appropriate manner and not merely in the specific combinations enumerated in the appended claims.

Exemplary embodiments 1-15 according to the second aspect of the invention:
1. A method for automated patient treatment sequencing, the method comprising the following steps:
   a) receiving medical data from at least one first medical device; and
   b) defining a patient stage within a predetermined patient treatment sequence, based at least in part on the medical data; and
   repeating the steps a) and b) for further medical data from the at least one first medical device and/or at least one second medical device.
2. The method of embodiment 1, wherein
   the at least one first and/or second medical device comprises an implant; and
   the method further comprises receiving medical data from at least one further device before implantation of the at least one first and/or second medical device, wherein defining the patient stage before implantation is based on the medical data from the at least one further device.
3. The method of embodiment 2, further comprising sending a data request to the at least one further device at least in part based on the medical data received from the at least one further device, from the at least one first medical device, and/or from the at least one second medical device.
4. The method of any of embodiments 1-3, further comprising validating the medical data, preferably by comparing the medical data to at least one predetermined criterion.
5. The method of any of embodiments 1-4, further comprising processing the medical data for a diagnostic identification of issues and/or for providing visualization data for visualization of the medical data.
6. The method of embodiment 5, wherein the type of processing is at least partly based on the stage of the patient.
7. The method of any of embodiments 5 or 6, further comprising forwarding the medical data for outputting to a user-interface.
8. The method of any of embodiments 1-7, further comprising pausing the method, based at least in part on the medical data indicating a pause condition, until receiving a continuation input.
9. The method of any of embodiments 1-8, further comprising terminating the method, based at least in part on the medical data indicating a termination condition.
10. The method of any of embodiments 1-9, wherein defining the patient stage further comprises accessing one or more predetermined criteria of the patient stage, and preferably further comprising determining whether the one or more predetermined criteria are met, at least in part based on the medical data.
11. The method of any of embodiments 1-10, wherein the repeating occurs regularly according to a predetermined repetition schedule.
12. The method of any of embodiments 1-11, further comprising decrypting the medical data from the at least one first and/or second medical device, preferably into a file structure with key-value pairs, more preferably in a human-readable language.
13. The method of any of embodiments 1-12, further comprising sending at least one reprogramming instruction to at least one of the first and/or second medical devices to switch from a first program to a second program; and preferably determining the patient stage based at least in part on the second program.
14. A computer program comprising instructions to execute the steps of the method of any of embodiments 1-13.
15. A system for automated patient treatment sequencing, the system comprising: means for receiving medical data from at least one first medical device;
   means for defining a patient stage within a predetermined patient treatment sequence, based at least in part on the medical data;
   means for receiving further medical data from the at least one first medical device and/or at least one second medical device; and
   means for defining the patient stage within the predetermined patient treatment sequence, based at least in part on the further medical data.

Exemplary embodiments 1-15 according to the third aspect of the invention:
1. A method for automated patient assistance, the method comprising:
   receiving medical data from an external device associated with a patient;
   defining a first patient stage within a predetermined patient treatment sequence, based at least in part on the medical data from the external device;
   receiving medical data from an implant of the patient; and
   defining a second patient stage within the predetermined patient treatment sequence, based at least in part on the medical data from the implant;
   wherein the medical data from the external device is received before implantation of the implant; and
   wherein the method further comprises providing educational material to the patient, based at least in part on the patient stage.
2. The method of embodiment 1, further comprising:
   registering the patient;
   defining the patient treatment sequence, comprising a plurality of stages, based at least in part on the registering.
3. A method for automated patient assistance, the method comprising:
   registering a patient;
   defining a patient treatment sequence, comprising a plurality of stages, based at least in part on the registering;
   receiving medical data associated with the patient;
   defining a patient stage within the patient treatment sequence, based at least in part on the medical data; and
   providing educational material to the patient, based at least partly on the patient stage.
4. The method of embodiment 3, further comprising:
   receiving medical data from an external device associated with a patient;
   defining a first patient stage within the predetermined patient treatment sequence, based at least in part on the medical data from the external device;
   receiving medical data from an implant of the patient; and
   defining a second patient stage within the predetermined patient treatment sequence, based at least in part on the medical data from the implant;
   wherein the medical data from the external device is received before implantation of the implant.
5. The method of any of embodiments 2-4, wherein the registering comprises a regional identification and/or a regulatory identification associated with the patient, wherein defining the patient treatment sequence is based at least in part on the regional identification and/or the regulatory identification.
6. The method of any of embodiments 1, 2, 4, or embodiment 5 when referred back to embodiment 2 or 4, wherein the educational material comprises at least one of: information about implant operation, information about implant settings, contact information of the implant manufacturer and/or involved clinicians, support information, troubleshooting information, and links to access any of this information.
7. The method of any of embodiments 1-6, wherein the educational material comprises at least one of: a video, a text, audio information, an image, a web-link, and a combination thereof.
8. The method of any of embodiments 1-7, wherein the patient treatment sequence comprises at least three patient stages, wherein at least partly different educational material is provided in each of the at least three stages.
9. The method of any of embodiments 1-8, further comprising outputting a notification on the patient stage and/or on the educational material for the patient.
10. The method of any of embodiments 1-9, further comprising forwarding the educational material for outputting to a user-interface.
11. The method of embodiment 10, wherein forwarding the educational material comprises forwarding the educational material in a preferred hierarchy, suggesting an order to the patient in which to view the educational material.
12. The method of any of embodiments 10 or 11, further comprising receiving a data request via the user-interface; and wherein forwarding the educational material is at least partly based on the data request.
13. A computer program comprising instructions to execute the steps of the method of any of embodiments 1-13.
14. A system for automated patient assistance, the system comprising:
   means for receiving medical data from an external device associated with a patient; means for defining a first patient stage within a predetermined patient treatment sequence, based at least in part on the medical data from the external device;
   means for receiving medical data from an implant of the patient;
   means for defining a second patient stage within the predetermined patient treatment sequence, based at least in part on the medical data from the implant; and
   means for providing educational material to the patient, based at least in part on the patient stage.
15. A system for automated patient assistance, the system comprising:
   means for registering a patient;
   means for defining a patient treatment sequence, comprising a plurality of stages, based at least in part on the registering;
   means for receiving medical data associated with the patient;
   means for defining a patient stage within the patient treatment sequence, based at least in part on the medical data; and
   means for providing educational material to the patient, based at least partly on the patient stage.

1. A system (5000) for medical data processing, the system comprising:
means for receiving (5170, 5180) encrypted medical data from a first secure medical device database (5030);
means for decrypting (5170, 5180) the medical data at least partly; and
means for providing the at least partly decrypted data to a second database (5050) accessible by a plurality of users via a web-based user interface.

2. The system (5000) of claim 1, wherein the means for decrypting (5170, 5180) are adapted such that one or both of:
the decrypted medical data comprises a file structure with key-value pairs, or
the decrypted data are free of privacy-sensitive information.

3. The system (5000) of claim 1, wherein the means for receiving (5170, 5180) is further configured to receive data from at least one external device (5090, 5270).

4. The system (5000) of claim 3, wherein the data comprise one or more of patient-reported data, health professional-reported data, or device-reported data.

5. The system (5000) of claim 1, further comprising means for triggering an alert, based at least in part on one or more of the medical data, or data received by the means for receiving (5170, 5180) from at least one external device (5090, 5270).

6. The system (5000) of claim 1, further comprising means for receiving a first data request from the second database (5050).

7. The system (5000) of claim 1, further comprising means for sending a second data request to the first secure medical device database.

8. The system (5000) of claim 7, further comprising means for providing medical data at least partly based on the second data request.

9. The system (5000) of claim 1, wherein the encrypted medical data comprise one or both of:
measurement data of an at least partly implantable medical device (5020), or
operational data of an at least partly implantable medical device (5020).

10. The system (5000) of claim 1, further comprising the secure medical device database, wherein the secure medical device database (5030) is configured to receive medical data from one or more medical devices.

11. The system (5000) of claim 10, further comprising means for data processing configured to automatically provide a follow-up report to the second database (5050) upon a program change of the one or more medical devices.

12. The system (5000) of claim 1, further comprising means for data processing configured for processing the medical data to one or both of:
diagnostically identify issues, or
provide visualization data for visualization of the medical data.

13. The system (5000) of claim 1, further comprising one or more of:
the second database;
the user-interface for outputting at least a part of the data in the second database (5050);
at least one medical device (5020); or
at least one relay device, wherein the relay device operably connects the at least one medical device (5020) and the secure medical device database (5030).

14. A method for medical data processing, the method comprising the following steps:
receiving encrypted medical data from a first secure medical device database (5030);
decrypting the medical data at least partly; and
providing the at least partly decrypted data to a second database (5050) accessible by a plurality of users via a web-based user interface.

15. A computer program comprising instructions to execute the steps of the method of claim 14.

16. The system (5000) of claim 2, the file structure with key-value pairs being in a human-readable language.

17. The system (5000) of claim 7, the second data request being based at least in part on a first data request received by a means for receiving from the second database (5050)
